(19) United States
(12) Patent Application Publication
(10) Pub. No.: US 2025/0239348 A1
(43) Pub. Date: Jul. 24, 2025

(54) FLUID THERAPY BASED ON PATIENT DATA, AND ASSOCIATED SYSTEMS, DEVICES, AND METHODS

(71) Applicant: REPRIEVE CARDIOVASCULAR, INC., Milford, MA (US)

(72) Inventors: Andrew HALPERT, Brookline, MA (US); Antony Jonathan FIELDS, San Francisco, CA (US); Dana COOK, Mequon, WI (US); Mark Richard PACYNA, Edina, MN (US); Cassandra E. MORRIS, Plymouth, MN (US)

(21) Appl. No.: 19/176,658

(22) Filed: Apr. 11, 2025

Related U.S. Application Data
(63) Continuation of application No. PCT/US23/76895, filed on Oct. 13, 2023.
(60) Provisional application No. 63/379,425, filed on Oct. 13, 2022.

(51) Int. Cl. G16H 20/10; A61B 5/20; G16H 10/60; G16H 50/70

(52) U.S. Cl. CPC G16H 20/10; A61B 5/208; G16H 10/60; G16H 50/70

(57) ABSTRACT

The present disclosure generally relates to fluid therapy based on patient data, and associated systems, devices, and methods. Embodiments of the present technology include trained models configured to improve the effectiveness of fluid therapy received by a patient. In some embodiments, the models improve a specific patient's response to fluid therapy, and predict whether a patient is expected to respond to fluid therapy, based on data associated with the patient's response to the fluid therapy.

### CROSS-REFERENCE TO RELATED APPLICATIONS

The present application is a continuation of International Patent Application No. PCT/US23/76895, filed Oct. 13, 2023, which claims priority to U.S. Provisional Patent App. No. 63/379,425, filed Oct. 13, 2022, the disclosures of which are incorporated herein by reference in their entireties.

### TECHNICAL FIELD

The present disclosure generally relates to medical devices and, in particular, to fluid therapy based on patient data, and associated systems, devices, and methods.

### BACKGROUND

Human physiological systems seek to naturally maintain a balance between fluid intake and fluid excretion. An imbalance in fluid intake and excretion rates may cause the body to retain excess amounts of fluid, also known as fluid overload. Fluid overload can be caused by acute decompensated heart failure (ADHF), chronic heart failure (CHF), or other conditions in which insufficient fluid is excreted. Patients exhibiting fluid overload may suffer from shortness of breath (dyspnea), edema, hypertension, and other undesirable medical conditions.

To treat fluid overload, patients are typically administered a diuretic drug which induces and/or increases urine production, thus reducing the amount of fluid and sodium in the body. The rate of urine output may be carefully monitored and/or controlled for safety reasons, e.g., to avoid placing undue stress on the patient's kidneys. Different patients may respond differently to treatment, such that the same diuretic type and/or dosage may produce drastically different urine output rates. However, conventional systems and methods for treating fluid overload may not be capable of accurately monitoring a patient's urine output and/or responding to changes in urine output. Additionally, conventional treatment systems and devices may not be capable of accommodating high urine production rates, and thus may require a nurse or other healthcare professional to empty and/or replace urine collection bags multiple times during the treatment procedure. Conventional systems and devices may also be prone to air lock and/or interruptions to urine flow.

### BRIEF DESCRIPTION OF THE DRAWINGS

Features, aspects, and advantages of the presently disclosed technology may be better understood with regard to the following drawings.

FIG. 1 is a partially schematic view of a fluid management system configured in accordance with embodiments of the present technology.

FIG. 2 is a flow diagram of a method for treating a patient, in accordance with embodiments of the present technology.

FIG. 3A is a block diagram that illustrates components of a fluid therapy treatment model system in accordance with embodiments of the present technology.

FIG. 3B is a block diagram that illustrates some of the components typically incorporated in at least some of the computer systems and other devices on which the disclosed systems can operate, in accordance with embodiments of the present technology.

FIG. 3C is a system diagram illustrating an example of a computing environment in which the disclosed systems can operate, in accordance with embodiments of the present technology.

FIG. 4A is a block diagram illustrating a method of building one or more models in accordance with embodiments of the present technology.

FIG. 4B is a block diagram illustrating a method of using one or more models in accordance with embodiments of the present technology.

FIGS. 5A-5C are block diagrams illustrating methods associated with diuretic dosing during fluid therapy, in accordance with embodiments of the present technology.

FIGS. 6A-6C are block diagrams illustrating methods associated with hydration fluid delivery during fluid therapy, in accordance with embodiments of the present technology

FIG. 7 is a block diagram illustrated a method associated with escalating a patient's fluid therapy in accordance with embodiments of the present technology.

FIGS. 8A-8D are block diagrams illustrating respective methods associated with ending a patient's fluid therapy, in accordance with embodiments of the present technology.

FIG. 9 illustrates a method associated with predicting and/or alerting users to one or more adverse events, in accordance with embodiments of the present technology.

FIG. 10 illustrates a method associated with one or more fluid levels of a fluid therapy system in accordance with embodiments of the present technology.

FIG. 11 illustrates a method associated with predicting a patient's urine output, in accordance with embodiments of the present technology

FIG. 12 illustrates a method associated with predicting an amount of one or more analytes in urine, in accordance with embodiments of the present technology.

FIG. 13 illustrates a method associated with predicting an outcome of fluid therapy for a patient, in accordance with embodiments of the present technology.

FIG. 14 illustrates a method associated with optimizing a net sodium loss rate of a patient, in accordance with embodiments of the present technology.

A person skilled in the relevant art will understand that the features shown in the drawings are for purposes of illustrations, and variations, including different and/or additional features and arrangements thereof, are possible.

### DETAILED DESCRIPTION

### I. Overview

The present technology is directed to systems for managing (e.g., increasing or decreasing) a patient's urine output based at least in part on an estimated excess fluid volume of the patient. Embodiments of the present technology relate to infusing diuretic and/or hydration fluid to increase or optimize urine output from the patient. While a standard treatment protocol can be effective for most patients, some patients can have unique conditions and/or have abnormal responses to the standard treatment protocols that prevent or inhibit optimal therapy. As an example, certain patients may not react to some diuretics and/or may have underlying conditions (e.g., low or high blood pressure) which limit their urine output rates, or make treatment to achieve maximum urine output rates more difficult. For such patients, additional steps or protocols may be necessary to increase urine output and relieve fluid overload conditions. These additional steps or protocols can be based on data associated with a patient receiving therapy, such as the patient's response to the received therapy (e.g., the administered diuretic and/or hydration fluid), and/or on historical treatment data including the treatment responses of one or more other patients. Accordingly, embodiments of the present technology are expected to optimize/customize all or a subset of a diuretic therapy to an individual patient's physiology, for example, to maximize decongestion and/or minimize clinical sequelae.

As described elsewhere herein, embodiments of the present technology can manage a patient's fluid removal based on the measured urine output and the physician's estimated excess fluid volume. For example, in some embodiments if a patient's urine output drops below a pre-defined rate and the patient has lost 80% or more of the estimated excess fluid volume or less than 1 L of estimated excess fluid remains to be removed from the patient, then the system may determine that therapy should be stopped (e.g., automatically stopped) immediately or after a period of time (e.g., one hour). Alternatively, if a patient's urine output drops below a pre-defined rate and less than 80% of the estimated excess fluid volume has been removed and/or more than 1 L of estimated excess fluid remains to be removed from the patient, then the system may determine that it is necessary to take steps to increase urine production. In such embodiments, the system may recommend (e.g., via software, labeling, etc.) infusing a second diuretic in addition to a first diuretic already being infused, and/or adjusting a rate of hydration fluid infusion. In doing so, embodiments of the present technology can advantageously manage a patient's urine output by ceasing one or more aspects of fluid therapy (e.g., diuretic infusion) for instances of sufficient fluid loss and improving fluid therapy by increasing urine output for instances of insufficient fluid loss.

Various aspects of one or more embodiments of the present technology can be based at least partially on one or more models (“model(s)”), such as artificial intelligence (AI) and/or machine learning (ML) models. Individual ones of these models can be trained using historical treatment data from one or more other patients and configured to determine and/or predict information about the patient receiving treatment, and/or otherwise inform the system's and/or the user's decisions regarding the patient's therapy. For example, in some embodiments the model(s) are configured to determine and/or predict information associated with diuretic delivery during fluid therapy. Individual ones of the model(s) can be configured to (i) predict the diuretic dose to elicit the desired urine output response from the patient, (ii) predict the occurrence of therapy re-ramps and/or automatically re-ramp the patient's therapy, and/or (iii) identify and/or select the diuretic(s) most likely to elicit the desired fluid removal response. Additionally, or alternatively, the model(s) are configured to determine and/or predict information associated with hydration fluid delivery during fluid therapy. For example, the model(s) can be configured to (i) predict the risk of the patient tolerating/not tolerating a given hydration fluid delivery rate, (ii) predict the likelihood of the patient's urine output exceeding a threshold value (e.g., falling below a threshold value), and/or (iii) adjust the patient hydration fluid delivery rate to improve the patient's urine output. In these and other embodiments, the model(s) are configured to determine and/or predict information associated with altering the patient's fluid therapy, including one or more steps, blocks, and/or protocols associated therewith. For example, the model(s) can be configured to (i) determine information associated with a decision to stop the patient's fluid therapy to guide a user's decision regarding the same, (ii) predict a readmission risk for the patient once the patient's fluid therapy has ended, (iii) recommend and/or determine oral diuretic dosage information associated with the patient, and/or (iv) identify patients not expected to respond to fluid therapy. In further embodiments, one or more of the model(s) are configured to determine, before beginning and/or during fluid therapy, a likelihood of the patient experiencing one or more adverse events during the fluid therapy. In some embodiments, the model(s) are configured to determine a time until a hydration fluid source and/or a diuretic source is expected to be empty and/or a time until a urine collection container is expected to be full.

In at least some embodiments, the model(s) can be configured/trained to compare patient data with training data and/or data associated with fluid therapy and/or other treatments administered to one or more other patients. For example, for a given patient, the model(s) can be configured/trained to identify data associated with one or more other patients that have an at least generally similar treatment profile. A treatment profile can be at least generally similar to data for a given patient if/when data associated with the treatment profile and the data for the given patient share one or more treatment characteristics (e.g., patient diuretic resistance, glomerular filtration rate (GFR), patient age, patient gender, overall patient health, underlying health conditions, renal function, demographics, clinician estimated excess fluid, etc.), urine rate profile (urine rate, slope of urine rate curve, plot of urine rate over time) an amount of fluid already removed from patient, a prior response to fluid therapy, etc. In some embodiments, rules for identifying an at least generally similar or identical treatment profile can be programmed into the model(s), and/or may correspond to values being within a range or threshold of one another (e.g., less than a 5%, 10%, 15%, 20%, 25%, or 30% difference). In other embodiments, the model(s) can identify/create rules, parameters, thresholds, etc. for identifying at least generally similar or identical treatment profiles, e.g., during training.

Generally, the model(s) are expected to improve the effectiveness of the fluid therapy steps/blocks/protocols described herein. In some embodiments, the model(s) are expected to improve a specific patient's response to the fluid therapy steps/blocks/protocols described herein. In further embodiments, the model(s) can predict whether a patient is expected to respond to fluid therapy and/or are expected to optimize (e.g., maximize) a patient's response to the fluid therapy steps/blocks/protocols described herein in real-time, for example, based on data received from the patient associated with the patient's response to the fluid therapy.

The headings provided herein are for convenience only and are not intended to limit or interpret the scope or meaning of the technology.

### II. Fluid Management Systems and Methods

The present technology is generally directed to systems, devices, and associated methods for fluid therapy based on patient data, including managing fluid levels of the patient based at least partially in response to data received from the patient before and/or during the fluid therapy. In some embodiments, the systems, devices, and methods described herein are used to treat a patient for fluid overload. To treat fluid overload, patients can be administered a diuretic drug which induces and/or increases urine production. For example, loop diuretics are diuretics that act at the ascending limb of the loop of Henle in the kidney, and include bumetanide (Bumex®), ethacrynic acid (Edecrin®), furosemide (Lasix®), torsemide (Demadex®), thiazide and thiazide-like diuretics (e.g., chlorothiazide, metolazone), potassium-sparing diuretics (e.g., amiloride, spironolactone), carbonic anhydrase inhibitors (e.g., acetazolamide), Vaptans (e.g., Conivaptan), SGLT2 inhibitors, and osmotic diuretics (e.g., mannitol). Diuretics can be given orally as a pill or as an intravenous (IV) injection. IV diuretics can be used when oral diuretics are no longer effective and/or able to be absorbed.

The short-term effects of diuretics on a patient's urine production may be difficult to predict, particularly at early stages of treatment. For example, one patient may produce much less urine than expected for a given dose of diuretic, while another patient administered the same dose may produce very large amounts of urine. Low urine production can prolong treatment time and/or reduce treatment efficacy, while high urine production can raise concerns of hypotension, hypovolemia, electrolyte imbalance (e.g., hypokalemia), and/or vital organ damage. High doses of a diuretic, regardless of the urine response, can also raise concerns about ototoxicity. Due to these uncertainties, physicians typically initially prescribe a conservative (e.g., low) diuretic dosage and wait a few hours before considering whether to increase the dosage. If the physician determines that a higher diuretic dosage is needed, the physician may slowly and incrementally increase the dosage until the patient's urine output reaches the desired level and/or rate. However, this approach can prolong the time the patient remains in the fluid overloaded condition, which can exacerbate the patient's underlying clinical state. For example, conservative treatment procedures can require hours or even days before the patient's urine output is sufficiently high to cause significant fluid loss and relieve the fluid overload condition. The patient may be hospitalized for several days (e.g., 4-5 days), which can be expensive and burdensome. Additionally, the long-term treatment efficacy may be limited, such that approximately 25% of patients are readmitted for fluid overload within 30 days.

To overcome these and other challenges, the present technology provides systems, and associated devices and methods, for managing a patient's fluid levels. In some embodiments, the present technology can (i) improve efficacy, safety, and quality of fluid management treatment, (ii) improve resource management in hospitals and other clinical settings, (iii) quickly assess if a patient is diuretic resistant, and/or (iv) increase diuretic efficiency (the amount of urine and/or excreted electrolytes (e.g., sodium) obtained over a given time per mg of diuretic infused intravenously). The embodiments described herein can increase net removal of fluid and/or electrolytes (e.g., sodium and/or chloride), and can also treat fluid overload conditions in a more efficient manner (e.g., shorter timeframe and/or higher net fluid loss).

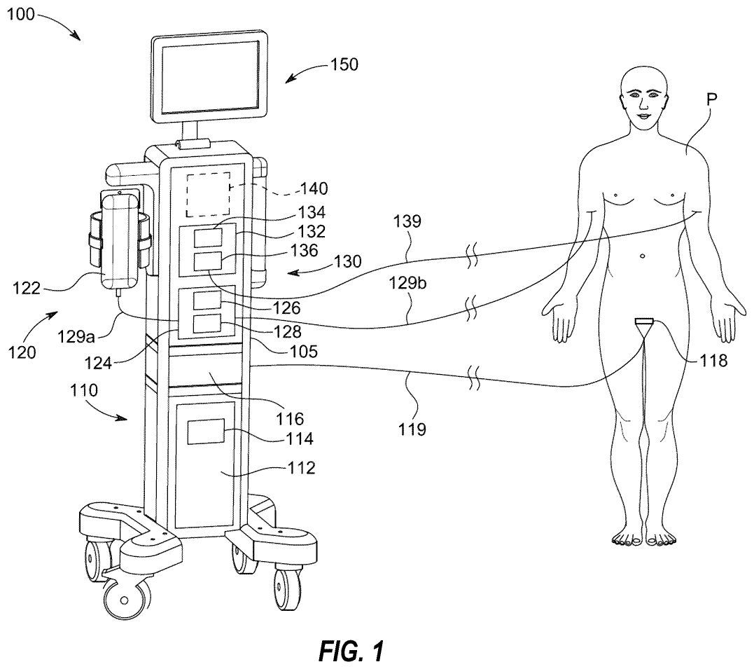

FIG. 1 is a partially schematic illustration of a fluid management system 100 (“system 100”) for monitoring urine output and/or control fluid infusion into a patient P, in accordance with embodiments of the present technology. The system 100 includes a urine collection and monitoring system 110 (“urine system 110”), an automated hydration fluid infusion system 120 (“hydration system 120”), an automated diuretic infusion system 130 (“diuretic system 130”), a controller or control system 140 (“controller 140”), and a display or input/output unit 150 (“display 150”). The controller 140 can be operably coupled to each of the urine system 110, hydration system 120, diuretic system 130, and/or display 150. The system 100 can further include a console or structure 105 (“console 105”) that incorporates, houses, and/or otherwise supports all or portions of the urine system 110, hydration system 120, diuretic system 130, the controller 140, and/or the display 150.

The urine system 110 is configured to collect urine from the patient P and/or monitor the patient's urine output (e.g., urine output amount and/or rates). The urine system 110 can include one or more collection containers 112 (“container 112”) configured to hold urine, such as a disposable bag or other collection device. The container 112 can be fluidly coupled to the patient P via a fluid line 119 (e.g., a tubing line). The fluid line 119 can be connectable to a disposable catheter 118 (e.g., a Foley catheter, Texas Condom catheter, PureWick catheter, etc.) placed in or otherwise connected to the bladder of the patient P.

In some embodiments, urine flow through the fluid line 119 is driven by the patient's urine production, gravity (e.g., the bladder of the patient P is positioned higher than the container 112), and/or a siphon effect between the patient's bladder and the container 112. In other embodiments, the urine system 110 can also include a pump (not shown) operably coupled to the fluid line 119 for actuating urine flow through the fluid line 119 and into the container 112. The pump can be or include any device suitable for pumping fluid, such as a peristaltic pump. The pump can be used to initiate urine flow from the patient's body at the start of the procedure. The pump can also be used to clear air locks and/or other obstructions from the fluid line 119.

The urine system 110 can include one or more sensors 114 (“sensor(s) 114”) configured to detect the patient's urine output (e.g., an amount and/or rate of urine output). The sensor(s) 114 can be operably coupled to the controller 140 so the controller 140 can monitor and/or compute the patient's urine output based on the data generated by the sensor(s) 114. The urine output can be determined in many different ways, such as based on urine flow (e.g., through the fluid line 119 and/or into the container 112), the amount of urine in the container 112 (e.g., based on the weight of the container 112, level of urine in the container 112, etc.), and/or other properties associated with the urine. The sensor(s) 114 can include one or more of the following: a flow sensor, drip counter, fluid weight sensor, fluid level sensor, float sensor, optical sensor, ultrasonic sensor, and/or other sensors known in the art suitable for measuring a urine output amount and/or rate. In the embodiment of FIG. 1, the sensor(s) 114 are positioned at the console 105. In other embodiments, however, some or all of the sensor(s) 114 can be at a different location in the system 100, such as on or in the line 119, on or in the container 112, and/or on or in the patient P.

In some embodiments, the sensor(s) 114 can include at least one sensor configured to measure one or more characteristics of the urine, in addition to detecting the patient's urine output. For example, the sensor(s) 114 can be configured to measure urine temperature, urine conductivity, urine oxygenation, urine specific gravity, and/or levels of one or more analytes in the urine (e.g., creatinine, sodium, potassium, etc.). Such characteristics can be useful, e.g., in determining effectiveness of a particular therapy and/or whether the patient P is in or could be approaching a critical condition. For example, urine conductivity and/or urine electrolytes (e.g., sodium) can indicate whether the patient is responding well to the fluid therapy, or whether the patient is in a critical condition and fluid therapy should cease. In some embodiments, urine conductivity (either alone or in combination with urine specific gravity) is used as a proxy for measurements of urine sodium and/or other urine electrolytes, e.g., a higher urine conductivity can correlate to higher urine sodium levels and a lower urine conductivity can correlate to lower urine sodium levels. As another example, urine temperature measurements can be used to detect urine flow (e.g., based on heat loss through the fluid line 119). The urine temperature can also be used as a proxy for the patient's body temperature, which in turn can correlate to the patient's current clinical state.

Optionally, the sensor(s) 114 can include at least one sensor configured to monitor the status of the urine collection procedure, such as whether urine collection is proceeding normally, whether there are interruptions in urine flow, whether there is a blockage or leak in the urine system 110, etc. For example, the sensor(s) 114 can include a leak sensor configured to detect whether a leakage is present in the urine system 110 (e.g., at or near the fluid line 119, catheter 118, and/or container 112). Leaks can be detected based on changes in urine flow rate, changes in pressure, the presence of moisture, or any other suitable parameter. In some embodiments, the controller 140 is configured to analyze the data from the leak sensor and/or other sensor(s) 114 to differentiate between low urine output rates versus leaks in the urine system 110.

As another example, the sensor(s) 114 can include a pressure sensor configured to measure the fluid pressure in the fluid line 119. The controller 140 can use the pressure measurements to monitor the status of urine flow, and optionally, detect whether there are any interruptions (e.g., decreases, sudden stoppages) or other issues with urine collection. In some embodiments, the controller 140 analyzes the pressure measurements to determine whether interruptions are due to low urine flow (e.g., the patient's bladder is empty or nearly empty), an air lock or other obstruction in the fluid line 119, a leak in the urine system 110 and/or a kink in the fluid line 119 and/or catheter 118. The controller 140 can alert the user if manual intervention is helpful or needed (e.g., to clear the obstruction, fix the leak, remove kinks from the fluid line 119, etc.). In embodiments where the urine system 110 includes a pump, the controller 140 can automatically activate the pump and/or increase the pumping rate to clear the obstruction from the fluid line 119.

The hydration system 120 can include at least one hydration fluid source 122 (“fluid source 122”-a bag, bottle, reservoir, etc.) containing a hydration fluid, such as saline (e.g., a premixed saline solution), Ringler's lactate solution, and/or other any other liquid solution suitable for infusion in the patient P. The hydration fluid can be isotonic, hypertonic, or hypotonic, e.g., depending on the patient's condition and/or other treatment considerations. Optionally, the composition of the hydration fluid (e.g., sodium, chloride, potassium, bicarbonate, etc.) can be varied based on the patient's condition and/or expected or measured electrolyte loss during the treatment procedure.

The fluid source 122 can be connected to the patient P via at least one fluid line (e.g., an IV line or other tubing), such as first fluid line 129a and a second fluid line 129b. The fluid source 122 can be operably coupled to one or more hydration fluid components 124 for actuating and/or monitoring hydration fluid infusion via the first and second fluid lines 129a-b, such as a hydration fluid pump 126 and/or at least one hydration fluid sensor 128 (“fluid sensor 128”). In the illustrated embodiment, the fluid source 122 is fluidly coupled to the hydration fluid pump 126 via the first fluid line 129a, and the hydration fluid pump 126 can pump the hydration fluid into the patient P via the second fluid line 129b. The hydration fluid pump 126 can be or include a peristaltic pump or other pump suitable for infusing a fluid into the patient's body (e.g., via an IV route or another route).

The fluid sensor 128 can be configured to determine an amount and/or rate of hydration fluid flowing from the fluid source 122 toward the patient P, and can include a flow sensor, pressure sensor, and/or other sensor configured to determine fluid output from the pump 126. Alternatively, or in combination, the fluid sensor 128 can monitor hydration infusion rate by measuring the pumping rate of the pump 126 (e.g., the number of rotations of the pump 126 per minute). As described elsewhere herein, the controller 140 can be operatively coupled to the hydration system 120 and can receive sensor data from the fluid sensor 128 to determine a hydration fluid infusion rate. The controller 140 can control the pumping rate of the pump 126 to control the amount and/or rate of hydration fluid provided to the patient P.

Optionally, the amount of hydration fluid in the fluid source 122 can be monitored, e.g., based on weight, volume, fluid levels, flow rates, etc. In such embodiments, the fluid source 122 can be operably coupled to an additional sensor separate from the fluid sensor 128 (not shown), such as a fluid level monitor, float sensor, weight sensor, optical sensor, drip counter, flow measurement sensor, or the like. The additional sensor can provide an independent source of measurement data for determining and/or verifying the amount and/or rate of hydration fluid being provided to the patient P, which can be helpful for improving measurement accuracy.

In some embodiments, the hydration system 120 includes at least one sensor configured to detect the presence of the fluid source 122, such as a location sensor, optical sensor, weight sensor, etc. The hydration system 120 can use the sensor data to automatically determine whether the fluid source 122 is present or absent, e.g., to assess whether the system 100 is ready to initiate the fluid therapy treatment. Optionally, the sensor data can be used to detect if the user is removing the fluid source 122 during the treatment procedure, e.g., to switch an empty or nearly empty fluid source 122 with a new fluid source 122. In such embodiments, the system 100 can automatically pause hydration fluid infusion until the fluid source 122 has been replaced. Accordingly, the user can switch fluid sources 122 without having to inform the system 100 or manually pause the procedure.

The diuretic system 130 can be configured to automatically provide a diuretic to the patient P. The diuretic system 130 can include a diuretic source 134 (e.g., syringe, bag, reservoir, etc.) containing a diuretic, such as bumetanide (Bumex®), ethacrynic acid (Edecrin®), furosemide (Lasix®), torsemide (Demadex®), and/or other diuretics known in the art, each of which may be part of a fluid solution (e.g., a mixture of saline and a diuretic or other agent). In some embodiments, the identity and/or concentration of the diuretic can be received by the controller 140 via user input (e.g., using the display 150), by scanning a barcode of the diuretic source 134 or other container of the diuretic, and/or any other suitable technique.

The diuretic source 134 can be connected to the patient P via a fluid line 139 (e.g., an IV line or other tubing). The diuretic source 134 can also be operably coupled to one or more diuretic components 136 for actuating and/or monitoring diuretic delivery via the fluid line 139. For example, the diuretic components 136 can include a diuretic pump configured to pump the diuretic through the fluid line 139 and toward the patient P. The diuretic pump can include a peristaltic pump, a syringe pump, a metering pump, or other device suitable for delivering the diuretic to the patient P at a plurality of dosage rates. The diuretic pump can deliver the diuretic according to any suitable delivery profile, such as at a controlled continuous rate and/or in controlled boluses delivered at regular intervals through the fluid line 139.

In some embodiments, the diuretic pump is or includes a syringe pump having a mechanical injector or plunger that is operably coupled to the controller 140, such that the controller 140 causes movement of the injector to transfer the diuretic to the patient P. The syringe pump can include or be coupled to an actuator that mechanically drives the injector to control the delivery of the diuretic to the patient P. For example, the actuator can be or include a mechanical actuator, such as a nut for rotating a screw to drive the injector. The syringe pump can also include or be operably coupled to a sensor for detecting the position of the injector. Alternatively, or in combination, the diuretic pump can include other types of pumps and/or actuators. For example, the diuretic pump can include a motor, a gearbox operatively connected to the motor, a sensor for measuring rotation of said motor (e.g., a tachometer or an optical encoder), and/or a microcontroller configured to control operation of the motor and monitor the quantity of diuretic delivered to the patient P. As another example, the diuretic pump can include an electric motor, such as a rotary motor, a linear motor, and/or a series of electrically actuated solenoids configured to propel liquid from the diuretic source 134 and through the line 139 toward the patient P.

In some embodiments, the diuretic components 136 include one or more diuretic sensors configured to determine an amount and/or rate of diuretic flowing toward the patient P. The one or more diuretic sensors can include, for example, a flow sensor, weight sensor, and/or other sensor type configured to determine the amount and/or rate of diuretic delivered from the diuretic source 134. Optionally, the diuretic sensors can measure diuretic delivery based on the output from the diuretic pump, such as by monitoring the pumping rate (e.g., number of rotations of the diuretic pump per minute, plunger position, etc.). The diuretic components 136 can include additional functional components, such as an air bubble detector, pressure sensor, extravasation sensor (e.g., ivWatch device), and/or other embedded electronics, e.g., to provide feedback signals to the controller 140 to ensure accurate diuretic infusion and/or monitor infusion status.

The controller 140 is configured to automatically control hydration fluid and/or diuretic infusion (e.g., based at least in part on the patient's urine output) to promote safe and effective diuresis of the patient P. The controller 140 can include one or more processor(s) and tangible, non-transient memory configured to store programmable instructions. The controller 140 can be operably coupled to the urine system 110, hydration system 120 and/or diuretic system 130 to receive data (e.g., sensor data) from and transmit data (e.g., control signals) to the various components of these systems. For example, the controller 140 can receive sensor data from the urine system 110 (e.g., from sensor(s) 114) to determine and/or monitor the patient's urine output. Based on the urine output, the controller 140 can determine an appropriate diuretic dosage amount and/or rate to administer to the patient P, and can cause the diuretic system 130 to deliver the diuretic accordingly. For example, the controller 140 can determine a pumping rate of the diuretic pump to produce the desired delivery profile for the diuretic. Similarly, the controller 140 can determine an appropriate hydration fluid infusion rate for the patient P (e.g., based on the urine output and/or the diuretic dosage rate), and can cause the hydration system 120 to deliver the appropriate hydration fluid amount and/or rate. For example, the controller 140 can determine a pumping rate for the hydration fluid pump 126 to achieve the desired hydration fluid infusion rate. The controller 140 can regulate the diuretic dosage rate and/or hydration fluid infusion rates based on a suitable treatment regimen protocol, e.g., prescribed by a physician and/or managed by the controller 140.

During the procedure, the controller 140 can receive sensor data from the various sensors of the urine system 110, hydration system 120 and/or diuretic system 130 to monitor the urine output, hydration fluid infusion rate, and/or diuretic dosage rate, respectively. The controller 140 can also receive sensor data from additional sensors configured to monitor patient status and/or operational status of the system 100, such as fluid pressure sensors, blood pressure sensors, air bubble detectors, analyte detectors, and the like. For example, the controller 140 can be operably coupled to at least one sensor implanted in, attached to, or otherwise associated with the patient P. The sensor(s) can provide data regarding any of the following patient parameters: pressure levels (e.g., pulmonary artery pressure, left atrial pressure), bioelectric measurements (e.g., bioimpedance vector analysis (BIVA)), hemoglobin measurements (e.g., non-invasive hemoglobin measurements), urine oxygenation levels, urine composition (e.g., creatine, sodium, potassium, chloride, etc.), urine temperature, body temperature (e.g., bladder temperature), oral fluid intake, heart rate, heart rate variability, blood oxygenation, hematocrit, hemodynamic data, and any other data described herein. The controller 140 can use the data from any of the sensors described herein to monitor treatment progress (e.g., whether the treatment is complete), patient status (e.g., whether the patient is responding well or poorly to treatment), and/or potential safety concerns (e.g., whether the diuresis is too aggressive, whether the patient is exhibiting side effects). The controller 140 can also adjust the hydration fluid infusion rate and/or diuretic dosage rate based on the sensor data. Additionally, the sensor data can also provide feedback to the controller 140 to confirm or verify the effectiveness of the fluid therapy.

The controller 140 can also use other data for monitoring and/or controlling the therapy, such as settings for the system 100, user input, data indicative of a desired treatment regimen (e.g., a programmed diuretic and/or hydration fluid delivery profile over time), and/or other data collected or calculated by the controller 140. In some embodiments, the data used by the controller 140 includes current and/or historical data for the patient P, such as diuretic dosages delivered to the patient P, urine output volume or rate, the amount of hydration fluid infused into the patient P, the weight or change in weight of the patient P at various times during the infusion of the diuretic, indicators of the patient's renal function (e.g., estimated glomerular Filtration Rate (eGFR)), and/or the time(s) during which the patient P was treated with the system 100. Additionally, or alternatively, the data used by the controller 140 can include historical data for one or more other patients, as described elsewhere herein.

The display 150 (e.g., a touchscreen, monitor, etc.) can include a user interface configured to receive inputs from the user and display outputs to the user. In some embodiments, the display 150 is operatively coupled to the controller 140 and thus can be used to receive user input indicating treatment parameters, such as parameters for urine output, hydration fluid infusion, and/or diuretic dosage. The treatment parameters can include, for example: a desired fluid balance level (e.g., a positive, negative, or neutral fluid balance), estimated excess fluid volume, target fluid removal volume (e.g., minimum and/or maximum amount of fluid to be removed), desired urine output level (e.g., a total amount of urine output; a target maximum, minimum, and/or average urine output rate), treatment duration (e.g., maximum and/or minimum duration of the treatment procedure; planned duration of the input balance level and/or urine output level), hydration fluid type, hydration fluid infusion rate (e.g., maximum, minimum, and/or average infusion rate), hydration fluid infusion profile (e.g., a function indicating how the amount and/or rate of hydration fluid infusion should vary over time), time limits associated with hydration fluid infusion (e.g., maximum and/or minimum time period for hydration fluid infusion), diuretic type, diuretic dosage (e.g., maximum and/or minimum dosage), diuretic dosage rate (e.g., maximum, minimum, and/or average dosage rate), diuretic dosage profile (e.g., a function indicating how the dosage amount and/or dosage rate of diuretic should vary over time), time limits associated with diuretic delivery (e.g., maximum and/or minimum time period for diuretic delivery), other fluids received by the patient during the procedure (e.g., volume of ingested fluid, volume of fluid from other medical agents besides the diuretic and/or hydration fluid), and/or suitable combinations thereof. Other patient-related inputs may also be received at the display 150 and can include, for example, the patient's sex, weight (e.g., “dry” weight), age, ethnicity, clinical state (e.g., renal function parameters, electrolyte levels such as serum chloride levels), medical history (e.g., outcomes of previous fluid removal procedures, prior response to fluid therapy, etc.), diagnoses (e.g., ADHF, CHF), medications (e.g., whether the patient is diuretic-naïve or diuretic-resistant), dietary factors (e.g., whether the patient is consuming a high-salt or low-salt diet, amount of oral fluid intake), etc.

Alternatively, or in combination, the user input via the display 150 can prompt the controller 140 to retrieve treatment parameters (e.g., maximum diuretic dosage, maximum continuous diuretic dosage, and minimum desired urine rate) from tables and/or other data sources. The data sources can be stored in the system 100 (e.g., in a memory associated with the controller 140) and/or can be stored in a separate device (e.g., a remote computing device). In some embodiments, the controller 140 retrieves data from a remote database and/or server via a communication network (e.g., a wired network, a wireless network, a cloud-based network, the Internet, and/or suitable combinations thereof). In such embodiments, the controller 140 can be operably coupled to a communication device and/or interface configured to transmit and receive data via the communication network.

The controller 140 can output the treatment parameters to the user via the display 150 for review and/or feedback. For example, the display 150 can show recommended treatment parameters for the patient P, such as recommendations for the diuretic, diuretic dosage rate (e.g., initial, maximum, and/or minimum dosage rate), hydration fluid infusion rate (e.g., initial, maximum, and/or minimum infusion rate), urine output rate (e.g., maximum and/or minimum output rate), treatment duration (e.g., maximum time period for diuretic and/or hydration fluid infusion; maximum total treatment duration), treatment escalation (e.g., thiazide, temporarily increased fluid matching, additional loop diuretic), end of treatment, and so on. As another example, the display 150 can output one or more predetermined treatment programs so the user can select the appropriate program for the particular patient P. Optionally, the user can modify any of the displayed treatment parameters, if desired.

During the treatment procedure, the controller 140 can output information regarding procedure status to the user via the display 150. For example, the controller 140 can display information regarding any of the following: urine output (e.g., current urine output rate and/or amount, urine output rate and/or amount over time, total amount of urine output so far), hydration fluid infusion (e.g., current infusion rate and/or amount, infusion rate and/or amount over time, total amount of hydration fluid infused so far), diuretic delivery (e.g., current dosage rate and/or amount, dosage rate and/or amount over time, total amount of diuretic delivered so far), fluid balance (e.g., current fluid balance, fluid balance over time, net fluid removal so far), system status (e.g., amount of hydration fluid remaining in the fluid source 122, amount of diuretic remaining in the diuretic source 134, remaining storage capacity in the container 112), treatment time (e.g., treatment start time, projected and/or planned treatment end time, total treatment duration so far), notifications (e.g., alerts, alarms, messages, recommendations, predictions, error messages), and the like. The user can review the displayed information, and, if appropriate, provide input instructing the controller 140 to adjust, pause, and/or stop the treatment procedure.

In some embodiments, the system 100 includes redundancy in the urine system 110, hydration system 120, and/or diuretic system 130 to reduce or minimize treatment interruptions, e.g., due to running out of urine collection capacity, running out of hydration fluid, and/or running out of diuretic. For example, the system 100 can include redundant components (e.g., containers 112, fluid sources 122, and/or diuretic sources 134), which can be stored at predetermined locations (e.g., on or within the console 105 or another portion of the system 100). The controller 140 can be configured to detect the presence of the redundant components, and can automatically or semi-automatically switch between these components so the treatment procedure can continue uninterrupted or substantially uninterrupted. Alternatively, or in combination, the system 100 can adjust the timing of user alerts related to urine collection capacity, hydration fluid levels, and/or diuretic levels, based on the availability of redundant components. For example, if redundant components are available, the system 100 can generate alerts at a later time (e.g., closer in time to when the container 112 would be full, when the fluid source 122 would be empty, and/or when the diuretic source 134 would be empty), since the system 100 can automatically switch to using the redundant components, or the user can rapidly perform the switch using the redundant components that are already stored locally at the system 100, rather than having to retrieve replacements from another location.

The lack of interruption in fluid therapy can help ensure effectiveness of the fluid therapy, e.g., by relieving the patient's fluid overload condition as quickly and safely as possible. In some embodiments, even brief interruptions in diuretic delivery and/or hydration fluid infusion can significantly affect the patient's urine output (e.g., cause the urine output rate to drop), which can interfere with therapeutic efficacy and prolong treatment time. The concerns described above regarding diuretic and/or hydration fluid backup supply may be unique to the present technology, e.g., due to the relatively large amounts of diuretic and/or hydration fluid that are utilized over time in some embodiments of the treatment procedures described herein. That is, whereas conventional systems and methods may utilize just a single diuretic source and/or a single hydration fluid source because of the relatively low amount of diuretic and/or hydration fluid administered, the present technology may benefit from multiple diuretic sources and/or hydration fluid sources to ensure treatment continuity. Similarly, the treatment procedures of the present technology can cause the patient P to produce relatively large volumes and/or rates of urine output compared to conventional procedures, such that multiple containers 112 may be helpful to reduce the number of times the user has to empty and/or replace the containers 112 during the procedure.

For example, in some embodiments, the urine system 110 includes two or more redundant containers 112 to ensure fluid therapy does not need to be stopped or interrupted due to the container 112 being full. In such embodiments, the urine system 110 can include a flow control assembly 116 (e.g., valves and/or other flow control components) operably coupled to the controller 140, and configured to selectively direct the urine from the patient P to one or more of the containers 112. The flow control assembly 116 can initially direct the urine received from the patient P to a first container 112. Once the flow control assembly 116 detects or determines the first container is full or nearly full (e.g., based on sensor data from the sensor(s) 114), the flow control assembly 116 can redirect the urine received from the patient P to a second container 112. While urine is being directed to the second container 112, a user can empty the first container 112 or replace the first container 112 with an empty container 112. The flow control assembly 116 and/or controller 140 can generate an alert to the user to indicate the first container is full and needs to be replaced or emptied. This process can be repeated such that fluid management therapy is not inadvertently interrupted due to the containers 112 being full and/or the urine system 110 being unable to accept urine output. In some embodiments, the treatment procedures described herein result in relatively large amounts and/or rates of urine output (e.g., compared to conventional therapies), such that automatic switching between multiple urine containers is advantageous to minimize treatment interruptions.

As another example, the hydration system 120 can include multiple redundant hydration fluid sources 122, e.g., to ensure the hydration fluid infusion can continue without interruption for the entirety of a therapy session and/or to provide an additional time window for switching hydration fluid sources 122 without interrupting hydration fluid infusion. In such embodiments, the hydration system 120 can include a hydration control assembly (e.g., valves and/or other flow control components-not shown) operably coupled to the controller 140, and configured to switch the source of hydration fluid from a first fluid source 122 to a second fluid source 122. In such embodiments, the hydration control assembly can initially deliver hydration fluid from the first fluid source 122 to the patient P. The hydration control assembly can monitor whether the first fluid source 122 is empty or nearly empty, e.g., based on data from the fluid sensor 128 and/or other sensors associated with the hydration system 120. Once the hydration control assembly detects or determines the first fluid source 122 is empty or nearly empty (e.g., the remaining amount of hydration fluid is below a predetermined threshold), the hydration control assembly can switch to delivering hydration fluid from the second source 122. The switching process can be repeated such that fluid therapy is not inadvertently interrupted due to the fluid source 122 being empty and/or the hydration system 120 being unable to provide hydration fluid.

The process of switching the hydration fluid source 122 can be performed automatically, semi-automatically, or manually. In some embodiments, semi-automatic or manual switching between the first and second fluid sources 122 may be beneficial to ensure the hydration system 120 does not automatically infuse hydration fluid without user confirmation. In such embodiments, the hydration control assembly and/or controller 140 can output an alert asking the user to verify that the hydration fluid should be switched from the first fluid source 122 to the second fluid source 122. Upon switching to the second fluid source 122, the controller 140 can generate an alert to the user to indicate the first fluid source 122 is empty and needs to be replaced. Optionally, the hydration control assembly and/or controller 140 can implement a pre-approval procedure in which the user allows the hydration system 120 to automatically infuse a specified volume of additional hydration fluid. Once that volume has been delivered to the patient P, the user may need to provide re-approval before further automatic infusion of hydration fluid.

In some embodiments, the different fluid sources 122 of the hydration system 120 each provide the same type of hydration fluid. In other embodiments, however, some or all of the fluid sources 122 can provide different types of hydration fluid. The hydration fluids can differ from each other with respect to tonicity, composition, electrolyte content, etc. Depending on the patient's response to diuresis, the hydration system 120 can deliver multiple different hydration fluids to the patient P sequentially or concurrently. For example, if the patient's urine output indicates that the patient P has an electrolyte imbalance (e.g., a positive sodium balance), the hydration system 120 can switch to delivering a hydration fluid that would address the imbalance (e.g., a hydration fluid with lower sodium content). The switching can be performed using any of the techniques and/or devices described above. Accordingly, the particular fluid or fluids delivered to the patient P can be tailored to the patient's particular clinical state and/or response to treatment.

In yet another example, the diuretic system 130 can include multiple redundant diuretic sources 134, e.g., to ensure the diuretic delivery can continue without interruption for the entirety of a therapy session and/or to provide an additional time window for switching diuretic sources 134 without interrupting diuretic delivery. For example, if a first diuretic source 134 (e.g., a first syringe or container) is spent, the diuretic can continue to be supplied (e.g., without substantial interruption) via a second diuretic source 134 (e.g., a second syringe or container). The second diuretic source 134 can be connected to the console 105, and can be operably coupled to a sensor configured to detect the presence of the second diuretic source 134 (e.g., a location sensor, optical sensor, weight sensor, etc.). Accordingly, the diuretic system 130 can switch to the second diuretic source 134 if the first diuretic source 134 is empty or nearly empty, and the second diuretic source 134 is present.

In some embodiments, the diuretic system 130 includes two independent diuretic pumps each including its own diuretic source 134. For example, the diuretic system 130 can include syringe pumps each fluidly coupled to its own syringe filled with diuretic. In some cases, such syringes may only be filled by pharmacists or other health care professionals, and thus may not be readily replaced (e.g., in less than a few hours) by the user. When the diuretic system 130 and/or controller 140 detects that the first diuretic source 134 is empty or nearly empty (e.g., below a predetermined threshold), the diuretic supply can be switched (e.g., automatically or manually) to a second diuretic source 134. The switching process can include stopping a first syringe pump fluidly coupled to the first syringe, and starting a second syringe pump fluidly coupled to the second syringe. In other embodiments, the diuretic system 130 includes a single diuretic pump (e.g., syringe pump) connected to two diuretic sources 134. In such embodiments, case switching between the first and second diuretic sources 134 can involve using a diuretic control assembly (e.g., valves and/or other flow control components) to switch the diuretic pump from delivering diuretic from the first diuretic source 134 to the second diuretic source 134. The switching process can be repeated such that fluid therapy is not inadvertently interrupted due to the diuretic source 134 being empty and/or the diuretic system 130 being unable to provide diuretic.

The process of switching the diuretic source 134 can be performed automatically, semi-automatically, or manually. In some embodiments, manual or semi-automatic switching between the first and second diuretic sources 134 may be beneficial to ensure the diuretic system 130 does not automatically infuse a large volume of diuretic without user confirmation. In such embodiments, the controller 140 can output an alert asking the user to verify that the diuretic should be switched from the first diuretic source 134 to the second diuretic source 134. Upon switching to the second diuretic source 134, the controller 140 can generate an alert to the user to indicate the first diuretic source 134 is empty and needs to be replaced. Optionally, the controller 140 can predict a time point and/or time range when the first diuretic source 134 will be empty (e.g., based on the diuretic dosage rate), and can output a notification so the user can order or otherwise prepare a replacement diuretic source 134 before the first diuretic source 134 runs out. Moreover, the diuretic control assembly and/or controller 140 can implement a pre-approval procedure in which the user allows the diuretic system 130 to automatically delivery a specified additional dosage of diuretic. Once that dosage has been delivered to the patient P, the user may need to provide re-approval before further automatic delivery of diuretic.

In some embodiments, the different diuretic sources 134 of the diuretic system 130 each provide the same type of diuretic. In other embodiments, however, some or all of the diuretic sources 134 can provide different types of diuretics. Depending on the patient's response to diuresis, the diuretic system 130 can deliver multiple different diuretics to the patient P sequentially or concurrently. For example, the diuretic system 130 can initially deliver a first diuretic to the patient P from a first diuretic source 134. If the patient P responds poorly to the first diuretic (e.g., the urine output rate does not increase or increases very slowly), the diuretic system 130 can switch to delivering a second, different diuretic from a second diuretic source 134. The diuretic system 130 can continue delivering the first diuretic concurrently with the second diuretic, or can terminate delivery of the first diuretic when the second diuretic is delivered. The switching can be performed using any of the techniques and/or devices described above. As another example, if the patient P does not respond well to a single diuretic, the diuretic system 130 can simultaneously administer multiple diuretics to the patient P. The ratio of the different diuretics can be varied as appropriate to elicit a suitable urine output rate. In other embodiments, however, rather than automatically administering additional diuretics, the diuretic system 130 can output a notification recommending that the user manually administer a different diuretic to the patient P and/or requesting that the user approve administration of a different diuretic, which may be beneficial for patient safety.

The system 100 illustrated in FIG. 1 can be configured in many different ways. For example, the locations of the various components of the system 100 can be altered, e.g., the urine system 110, hydration system 120, and/or diuretic system 130 can be at different locations in the console 105. As another example, any one of the urine system 110, hydration system 120, or diuretic system 130 can be part of a separate system or device (e.g., a separate console), or can be omitted altogether. For instance, in some embodiments, the urine system 110 is replaced with a mechanism for monitoring the patient's urine output that does not require the catheter 118 and/or urine collection, such as an ultrasound sensor that measures the patient's bladder volume. The ultrasound sensor can be implemented as a patch or similar device that is coupled to the patient's body. The controller 140 can process the ultrasound sensor data to detect changes in the bladder volume, and can determine the corresponding amount and/or rate of urine output based on the bladder volume. The use of non-invasive urine monitoring mechanisms such as an ultrasound sensor can allow the treatment procedures described herein to be performed in outpatient settings.

As another example, in some embodiments, the hydration system 120 is omitted such that diuresis is performed without hydration fluid infusion, or the hydration fluid is infused manually. Diuresis with hydration fluid infusion may be more beneficial for patients with low serum chloride levels (e.g., patients with low-salt diets), while patient with high serum chloride levels (e.g., patients with high-salt diets) may tolerate diuresis with little or no hydration fluid infusion. Optionally, the hydration fluid infusion rate can be varied at least partially based on the patient's serum chloride levels, e.g., lower amounts and/or rates of hydration fluid infusion can be used if the patient's serum chloride level is high (e.g., greater than or equal to 105 mmol/L).

In yet another example, the diuretic system 130 can be omitted such that no diuresis is performed, or the diuresis is performed manually. In such embodiments, the system 100 can provide automated fluid replacement via the hydration system 120 and/or can automatically monitor the patient's urine output via the urine system 110, but the diuretic would be administered manually by a healthcare professional in accordance with techniques known to those of skill in the art.

The system 100 can optionally include or be used in combination with additional systems or devices, such as systems or devices configured to perform any the following functions: administering other medications and/or agents besides the diuretic and hydration fluid (e.g., heart failure medication), monitoring other patient parameters besides urine output (e.g., blood pressure, weight, heart rate, blood oxygenation, respiratory rate, temperature), and/or performing other types of medical procedures on the patient P concurrently or sequentially with the fluid removal procedure (e.g., dialysis, ultrafiltration).

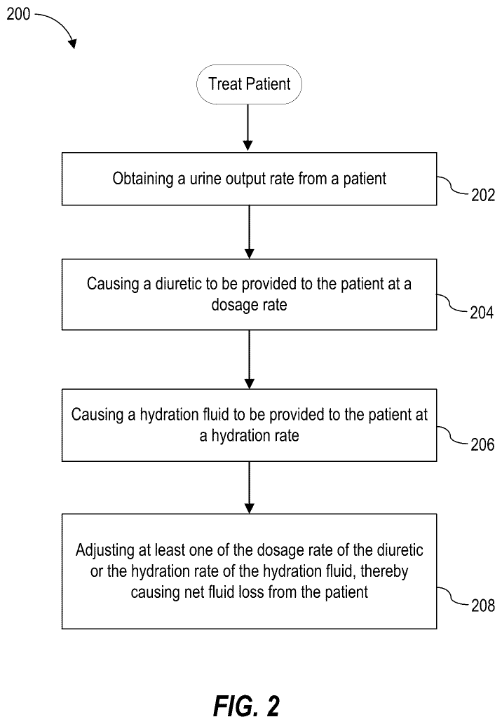

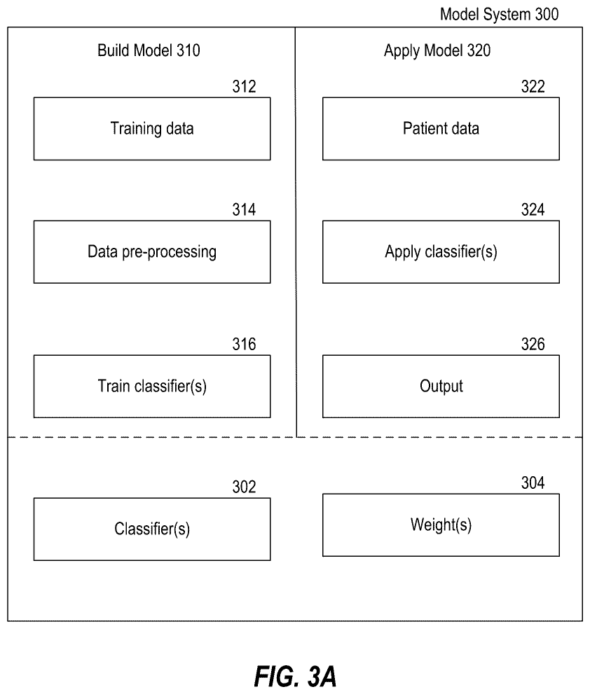

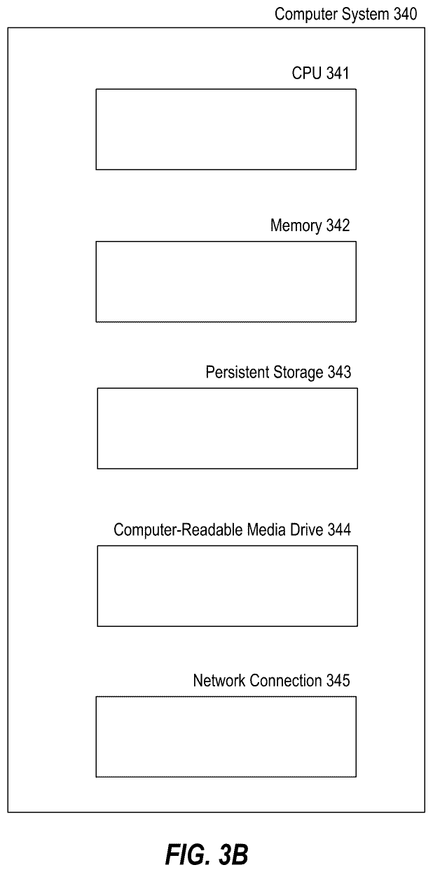

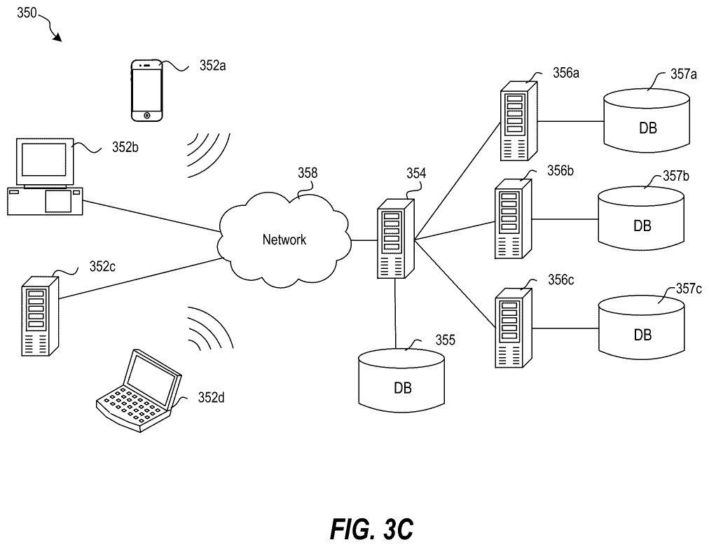

FIG. 2 is a flow diagram of a method 200 for treating a patient, in accordance with embodiments of the present technology. In some embodiments, the method 200 is used to treat the patient for fluid overload by removing fluid from the patient to produce a negative fluid balance (net fluid loss). The method 200 can be performed by any embodiment of the systems and devices described herein, such as the system 100 of FIG. 1. In some embodiments, some or all of the blocks of the method 200 are performed by a system or device including one or more processors and a memory storing instructions that, when executed by the one or more processors, cause the system or device to perform one or more of the blocks described herein. For example, the method 200 can be performed by the controller 140 of the system 100 of FIG. 1, CPU 341 of FIG. 3B, and/or another suitable processor. Optionally, some or all of the blocks of the method 200 can performed automatically or semi-automatically, with little or no human intervention.

The method 200 can begin at block 202 with obtaining a urine output rate from a patient. The urine output rate can be obtained from a urine monitoring and/or collection system connected to the patient, such as the urine system 110 of FIG. 1. The system can determine the urine output rate based on received input data, such as data from one or more sensors (e.g., the sensor(s) 114 of FIG. 1). As described above, the sensor(s) can be configured to measure the urine output rate based on flow rate, weight (e.g., of the container 112 of FIG. 1), volume, fluid level, and/or any other suitable parameter. The urine output rate can be calculated based on the received input, e.g., by a controller (e.g., controller 140 of FIG. 1) operatively coupled to the sensor(s). The urine output rate can be a current rate or an average rate measured over a predetermined time period (e.g., the previous 5 or 10 minutes). The urine output rate can be updated on a continuous or recurring basis (e.g., every 30 seconds, 1 minutes, 2 minutes, etc.). In some embodiments, the process of block 202 is performed concurrently with some or all of the other blocks of the method 200 (e.g., blocks 204, 206, and/or 208) to provide continuous or substantially continuous urine output monitoring through the entirety of the method 200.

At block 204, the method 200 optionally continues with causing a diuretic to be provided to the patient at a dosage rate. The diuretic can be or include furosemide, bumetanide, ethacrynic acid, torsemide, combinations thereof, and/or other diuretics known in the art. In some embodiment, the diuretic is delivered as part of a solution including saline or other hydration fluid(s) mixed therewith. The diuretic can be provided automatically or semi-automatically by a diuretic system connected to the patient, such as the diuretic system 130 of FIG. 1. The diuretic system can be operably coupled to a controller (e.g., controller 140 of FIG. 1) for causing diuretic delivery in accordance with a planned and/or pre-programmed treatment procedure.

In some embodiments, the treatment procedure includes multiple phases, and each phase is associated with a different delivery profile for the diuretic. In such embodiments, block 204 can be performed as part of an initial phase to determine an appropriate diuretic dosage rate for treating the patient (also known as a “dosage determining phase”). In the dosage determining phase, the diuretic is injected at an initial dosage rate, and the dosage rate can then be gradually increased (e.g., “ramped”) to elicit an increase in the patient's urine output rate. The diuretic dosage rate can be increased according to a desired function or delivery profile, such as a continuous function, a block-wise function, or a combination thereof). The function can include iteratively increasing the dosage rate linearly, exponentially, according to a polynomial function, and/or any other suitable ramp function or profile. In some embodiments, the diuretic is delivered in a manner such that a subsequent dosage rate is a predetermined percentage (e.g., at least 5%, 10%, 15%, 25%, etc.) above the immediately previous dosage rate. The predetermined percentage can increase or decrease over time, e.g., depending on the desired fluid therapy and/or patient considerations. Optionally, the diuretic can be provided in a manner that doubles the diuretic dosage rate or total diuretic within a period of time (e.g., 10 minutes, 15 minutes, 20 minutes, or within a range of 10-20 minutes). In other embodiments, however, the dosage determining phase can include one or more time periods during which the diuretic dosage rate does not increase and/or is held substantially constant. The dosage determining phase can continue until the patient's urine output reaches or exceeds a desired threshold rate and/or a predetermined time period has elapsed, at which point the diuretic dosage rate can be adjusted, as described in block 208 below.

At block 206, the method 200 can optionally include causing a hydration fluid to be provided to the patient at a hydration rate. The hydration fluid can comprise saline and/or other fluids having sodium, and can be provided automatically or semi-automatically by a hydration fluid system connected to the patient, such as the hydration system 120 of FIG. 1. The hydration fluid can be provided before, during, and/or after providing the diuretic in block 204 (e.g., before, during, and/or after the dosage determining phase). Intravenous infusion of hydration fluid containing electrolytes (e.g., sodium and/or chloride) can increase diuretic efficiency, which is counterintuitive since a goal of fluid therapy is net removal of fluid. Hydration fluid can also reduce or inhibit intravascular depletion, decreases in cardiac output, and/or decreases in renal perfusion, among other benefits.

In some embodiments, the hydration fluid is provided to the patient based at least in part on the corresponding urine output rate, e.g., to drive net fluid loss from the patient. For example, the hydration rate can be less than the urine output rate. In some embodiments, the hydration rate is a percentage of the urine output rate (e.g., 90%, 80%, 70%, 60%, 50%, 40%, 30%, 20%, or 10% of the urine output rate) for a given range of urine output rates (e.g., from 0 ml/hr to 1000 ml/hr). Optionally, the percentage can be higher for certain parts of the range (e.g., for the lower end of the range to reduce the likelihood of hypotension) and/or lower for other parts of the range (e.g., for the higher end of the range to increase net fluid loss). As another example, the hydration rate can substantially match the urine output rate (e.g., 100% of the urine output rate) for an initial amount of urine output by the patient (e.g., at least the initial 150 ml, 200 ml, or 250 ml), for an initial time period (e.g., the first hour, 2 hours, or 3 hours), for an initial time period during hydration fluid and/or diuretic dose finding, and/or until the patient's urine output rate reaches a predetermined threshold. Subsequently, the hydration rate can be adjusted to be less than the urine output rate. In a further example, the hydration rate may be determined based on whether the urine output rate is above or below one or more different thresholds, with the difference between the urine output rate and hydration fluid rate increasing as the urine output rate increases. In such embodiments, the difference between the urine output rate and the hydration fluid rate can increase (with the urine rate being higher than the hydration fluid rate) as the urine output rate increases, and thus the net fluid loss from the patient can increase as the urine output rate increases.

At block 208, the method 200 can include adjusting at least one of the dosage rate of the diuretic or the hydration rate of the hydration fluid, thereby causing net fluid loss from the patient. For example, the (i) diuretic dosage rate can be adjusted, (ii) the hydration rate can be adjusted, or (iii) the diuretic dosage rate and the hydration rate can both be adjusted. In some embodiments, the diuretic dosage rate is adjusted after the dosage determining phase of the treatment procedure is complete. As discussed above in block 204, the dosage determining phase can end when (i) a predetermined amount of time has elapsed since the initial diuretic administration, and/or (ii) the urine output rate is or becomes greater than or equal to a predetermined threshold rate. The treatment procedure can then switch to a phase in which the diuretic dosage rate is adjusted to a dosage rate configured to maintain the patient's urine output rate at or above a desired output rate to cause net fluid loss (also known as a “continuous delivery phase” or “fluid reduction phase”).

The adjusted diuretic dosage rate can be the initial dosage rate for the fluid reduction phase, and can be determined in many different ways. For example, the adjusted diuretic dosage rate can be based on the outcome of the dosage determining phase (e.g., the dosage rate when the patient's urine output reaches or exceeds the target threshold). In representative embodiments, the diuretic dosage rate is decreased, e.g., to maintain the patient's urine output rate at a predetermined rate and/or within a predetermined range (e.g., no more than 5%, 10%, or 20% variability from a predetermined rate). Decreasing the diuretic dosage rate can decrease the rate of increase in urine output rate (e.g., cause the patient's urine output to approach a constant or substantially constant rate) but without actually decreasing the urine output rate itself.

In some embodiments, the adjusted diuretic dosage rate is a predetermined percentage or fraction of the current dosage rate (e.g., the dosage rate at the end of the dosage determining phase) or a predetermined percentage of the cumulative diuretic dosage amount (e.g., the cumulative amount delivered during the dosage determining phase). For example, the adjusted dosage rate can be a predetermined percentage (e.g., 10%, 15%, 20%, 25%, 30%, or within a range of 10-30%) of a value of the total amount of diuretic delivered to the patient at that time. For example, if the total amount delivered is 100 mg, and the predetermined percentage is 25%, then the adjusted dosage rate can be 25 mg/hr. In some embodiments, the percentage used to calculate the adjusted diuretic dosage rate is based on a pharmacokinetic characteristic of the particular diuretic being infused. For example, the percentage can be 20% for furosemide, such that if 50 mg of furosemide is infused in 60 minutes, then the adjusted diuretic dosage rate can be 10 mg/hr.

In some embodiments, block 208 includes delivering the diuretic at the adjusted diuretic dosage rate until the fluid reduction phase is complete, e.g., until a predetermined period of time has elapsed, the patient's urine output drops below a low urine output threshold, and/or until a target net fluid loss volume is achieved. During the fluid reduction phase, the diuretic dosage rate can be constant or substantially constant (e.g., no more than 5%, 10%, or 20% variability from the initially determined adjusted diuretic dosage rate). In other embodiments, however, block 208 can include making additional adjustments to the diuretic dosage rate during the treatment procedure (e.g., increasing and/or decreasing the diuretic dosage rate). The adjustments can be based on whether one or more of a predetermined set of conditions is met, such as whether the urine output rate is too high (e.g., which can indicate that the patient has a high and/or increasing serum level of diuretic). The set of conditions can include (i) an average urine rate being greater than a predetermined rate for a period of time, (ii) an average rate of change of the urine rate being greater than a predetermined rate of change, and/or (iii) a diuretic dosage rate being greater than a predetermined dosage rate. If some (e.g., two) or all of the conditions are met, the diuretic dosage rate can be decreased (e.g., by a predetermined amount or percentage), also referred to herein as “down-titration.”

In some embodiments, a down-titration is performed only if all or a majority of the above conditions are met, which can avoid unnecessarily decreasing the diuretic dosage rate, thereby allowing urine output rates to remain high and avoiding unnecessary interruptions to the treatment procedure. For example, whereas other methodologies may interrupt fluid therapy and decrease the diuretic dosage rate (e.g., to zero mg/hr) only when the urine rate too high, the process described herein may only decrease the dosage rate (e.g., to a non-zero or zero dosage rate) when one or more factors are met, such as when the urine output rate is both high and continuing to increase. Stated differently, the process herein can prevent the diuretic dosage rate from being unnecessarily decreased when urine rates are temporarily high (e.g., above the predetermined rate), but are trending downward. This approach can prevent or inhibit over-diuresis, excess fluid loss and/or electrolyte loss, as well limit unnecessary exposure of the patient to additional diuretic. Additionally, because the diuretic dosage rate can be down-titrated, rather than stopping the diuretic entirely, the fluid therapy can continue (albeit at lower urine output rates) without needing to completely restart the procedure.

As another example, the additional adjustments to the diuretic dosage rate in block 208 can include increasing the diuretic dosage rate, also referred to herein as “re-ramping” or “up-titration.” In some embodiments, re-ramping is performed if urine output rates are too low, as determined based on a set of conditions. The set of conditions can include (i) the average urine rate being below a predetermined threshold rate for a predetermined period of time, and/or (ii) more than a predetermined amount of debt has accumulated over the predetermined period of time. “Debt” can be defined as the area on a plot between the urine output rate and a set rate (e.g., 325 ml/hr), and can represent how much of and for how long the urine output rate has been below the set rate. If some or all of the conditions are met, re-ramping can be performed by incrementally increasing the diuretic dosage rate until (i) a predetermined amount of time has elapsed, and/or (ii) the urine output rate is or becomes greater than or equal to a predetermined threshold rate. The re-ramp process can be identical or generally similar to the dosage determining process previously described in block 204. In representative embodiments, the dosage rate or “ramp” can start at any dosage identified during the dosage determining process, such as the current dosage rate, a previously determined dosage rate, or another suitable dosage rate (e.g., not at the beginning of the dosage).

The re-ramping process can be performed automatically, semi-automatically, or manually. In some embodiments, re-ramping is a semi-automatic or manual process requiring user approval, e.g., for regulatory and/or safety reasons. In such embodiments, the system can output a notification to the user (e.g., via the display 150 of FIG. 1) instructing the user to confirm that re-ramping should be initiated. Optionally, the system can implement a pre-approval procedure in which the user can allow the system to automatically perform re-ramping under certain conditions (e.g., within a specific time period, until a certain urine output volume and/or rate is achieved, for a maximum diuretic amount and/or dosage rate, etc.). This approach can allow for automatic re-ramping under limited circumstances, which can reduce the amount of human intervention during the treatment procedure and improve the responsiveness of the system to the patient's current state. Once the pre-approval conditions have elapsed, the user may need to provide re-approval before additional automatic re-ramping is allowed.

In some embodiments, block 208 also includes adjusting the diuretic dosage rate in response to a potential and/or detected blockage (e.g., an air lock, a kink in a fluid line, etc.) in the urine collection system. For example, an air lock can be any partial or complete obstruction of fluid flow due to trapped gas (e.g., air) within a fluid system. Air locks may produce an artificial drop in urine output rates, which can affect the determination of the diuretic dosage rate (e.g., result in a diuretic dosage rate that is too high). In some embodiments, the presence of an air lock is detected based on a period of little or no urine output (due to the air lock blocking urine flow), followed by a sudden large bolus of urine output (due to built-up pressure in the fluid line clearing the air lock). When the system detects that an air lock or other blockage was or is present, the system can compensate by adjusting the diuretic dosage rate to the dosage rate that should have been used if the air lock or other blockage had not occurred. The appropriate dosage rate can be determined based on historical data for the patient receiving the fluid therapy and/or one or more other patients (e.g., the diuretic dosage rate before the air lock occurred, a diuretic dosage rate calculated from the patient's urine output rate before the air lock occurred, etc.).

Alternatively, or in combination, block 208 can include adjusting the hydration rate, e.g., by increasing or decreasing the hydration rate based on the patient's urine output rate to drive net fluid loss from the patient. For example, as previously described, the hydration rate can initially match the patient's urine output rate for a set of initial conditions (e.g., certain time period, initial urine output amount, and/or initial urine output rate). Once the initial conditions have elapsed, the hydration rate can be maintained at a rate lower than the urine output rate (e.g., a percentage of the urine output rate) so the patient exhibits net fluid loss during the fluid reduction phase. The hydration rate can be determined in various ways, such as a percentage or fraction of the patient's urine output rate, based on whether the urine output rate is above or below a number of different thresholds (e.g., with the difference between the urine output rate and hydration rate increasing as the urine output rate increases), and/or any other suitable approach.

Optionally, the diuretic dosage rate and/or hydration rate can be adjusted based on factors other than patient's urine output rate. For example, the diuretic dosage rate and/or hydration rate can be adjusted based on the patient's blood pressure in order to avoid placing the patient in a hypotensive state. In some embodiments, if the patient's blood pressure level is too low (e.g., below a threshold value or range), the system can avoid increasing the diuretic dosage rate and/or can decrease the diuretic dosage rate for a certain period of time. Alternatively, or in combination, the system can increase the hydration rate (e.g., to the maximum allowable hydration rate and/or to provide a desired fluid replacement profile (e.g., a 100% match to the patient's urine output rate)) for a certain period of time if low blood pressure levels are detected. The system can also output an alert indicating that the patient's blood pressure level is low so a user can check on the patient's status. Optionally, the system can take both blood pressure levels and urine output rates into account, e.g., the system can generate alerts and/or can adjust the diuretic dosage rate and/or hydration rate if the Patient's blood pressure is low and the patient's urine output rate drops. This approach can improve patient safety and control over the treatment procedure.

In some embodiments, some or all of the blocks of the method 200 are performed as part of a medical procedure for treating the patient for a fluid overload condition. The method 200 can be used as a primary, standalone therapy for treating fluid overload, or can be used in combination with other therapies (e.g., as a post-primary therapy to reduce the likelihood of re-hospitalization). The method 200 can be performed in any suitable setting, such as an inpatient setting or an outpatient setting. In embodiments where the method 200 is performed as an outpatient therapy, the overall duration of the method 200 can be reduced (e.g., to no more than 10 hours, 5 hours, 4 hours, 3 hours, 2 hours, or 1 hour).

The method 200 illustrated in FIG. 2 can be modified in many different ways. For example, any of the blocks of the method 200 can be omitted, such as blocks 204 or 206. In some embodiments, block 204 is omitted so that the method 200 controls hydration fluid infusion but not diuretic delivery, or so that the method 200 does not involve any diuretic delivery at all. Similarly, block 206 can be omitted so that the method 200 controls diuretic delivery but not hydration fluid infusion, or so that the method 200 does not involve any hydration fluid infusion at all. As another example, some or all of the blocks 200 of the method 200 can be performed in a different order and/or repeated (e.g., any of blocks 202, 204, 206, and/or 208). In a further example, the method 200 can optionally include additional blocks not shown in FIG. 2 (e.g., causing delivery of additional medications, obtaining parameters other than urine output rate, etc.).

The present technology can provide many advantages for treating fluid overload and/or managing patient fluid levels. For example, embodiments of the present technology have been shown to consistently reduce the fluid volume in patients faster and safer than conventional treatment systems and methods. For example, whereas conventional methods can typically take at least five days to remove 4-5 L of net fluid volume, embodiments of the present technology have been shown to remove 4-5 L liters of net fluid volume in no more than 24 hours. Additionally, embodiments of the present technology have also been shown to remove significant amounts of salt via high sodium urine from patients. This can reduce the likelihood of the patient reaccumulating fluid after discharge, which can lead to reductions in rehospitalization rates. Moreover, embodiments of the present technology can automatically and continuously monitor urine output, hydration fluid infusion, and/or diuretic delivery to mitigate patient safety concerns (e.g., over-diuresis and/or hypotension) during the treatment procedure.

Embodiments of the present technology can provide various benefits, such as any of the following: (i) optimizing net fluid volume removal; (ii) reducing the time needed to achieve desired net fluid removal by allowing physicians to use higher diuretic dosages and/or dosage rates earlier in treatment compared to conventional treatments; (iii) avoiding or reducing risk of adverse events such as over-diuresis, dehydration, and/or intravascular depletion; (iv) quickly assessing if a patient is diuretic resistant; and (v) providing a record of treatment data. Embodiments of the present technology may obtain an average net fluid removal rate (e.g., average urine output rate minus average hydration fluid infusion rate) of at least 225 ml/hr, which provides 3.4 L per day of net fluid volume removal based on introducing 2 L of fluid per day orally or through IV infusion. This rate of fluid removal, while replacing sodium, may reduce the overall length of stay and/or provide enhanced decongestion.

FIG. 3A is a block diagram that illustrates components of a fluid therapy treatment model system 300 (“model system 300”) in accordance with embodiments of the present technology. The model system 300 can include one or more classifiers 302 configured to train and/or build one or more machine learning and/or artificial intelligence models. The classifiers 302 may include a variety or combination of classifiers including neural networks, such as a fully-connected, convolutional, recurrent, autoencoder (e.g., a restricted Boltzmann machine), a support vector machine, a Bayesian classifier, and so on. When the classifier is a deep neural network, the training results in a set of weights 304 for the activation functions of the deep neural network. A support vector machine (“SVM”) operates by finding a hyper-surface in the space of possible inputs. The hyper-surface attempts to split the positive examples (e.g., feature vectors associated with positive and/or improved patient outcomes) from the negative examples (e.g., feature vectors associated with negative, worsened, and/or unimproved patient outcomes) by maximizing the distance between the nearest of the positive and negative examples to the hyper-surface. This block allows for correct classification of data that is similar to but not identical to the training data. Various techniques can be used to train a support vector machine.

Adaptive boosting is an iterative process that runs multiple tests on a collection of training data. Adaptive boosting transforms a weak learning algorithm (an algorithm that performs at a level only slightly better than chance) into a strong learning algorithm (an algorithm that displays a low error rate). The weak learning algorithm is run on different subsets of the training data. The algorithm concentrates more and more on those examples in which its predecessors tended to show mistakes. The algorithm corrects the errors made by earlier weak learners. The algorithm is adaptive because it adjusts to the error rates of its predecessors. Adaptive boosting combines rough and moderately inaccurate rules of thumb to create a high-performance algorithm. Adaptive boosting combines the results of each separately run test into a single, very accurate classifier. Adaptive boosting may use weak classifiers that are single-split trees with only two leaf nodes.

A neural network model has three major components: architecture, cost function, and search algorithm. The architecture defines the functional form relating the inputs to the outputs (in terms of network topology, unit connectivity, and activation functions). The search in weight space for a set of weights that minimizes the objective function is the training process. In one embodiment, the classification system may use a radial basis function (“RBF”) network and a standard gradient descent as the search technique.

In some embodiments, the model system 300 may use various design-of-experiments (“DOE”) techniques to identify values of feature vectors of consumer entities that result in positive outcomes for various action inducers. Suitable DOE techniques include central composite techniques, Box-Behnken techniques, random techniques, Plackett-Burman techniques, Taguchi techniques, Halton, Faure, and Sobel sequences techniques, Latin hypercube techniques, and so on. (See Cavazzuti, M., “Optimization Methods: From Theory to Design,” Springer-Verlag Berlin Heidelberg, 2013, chap. 2, pp. 13-56, which is hereby incorporated by reference.) The Latin hypercube technique has the characteristic that it generates sample values in which each axis (i.e., feature) has at most value that is selected.

In some embodiments, the model system 300 may use by a Convolutional Neural Network (“CNN”). A CNN has multiple layers such as a convolutional layer, a rectified linear unit (“ReLU”) layer, a pooling layer, a fully connected (“FC”) layer, and so on. Some more complex CNNs may have multiple convolutional layers, ReLU layers, pooling layers, and FC layers. The training data component 312 can input training data (e.g., that may or may not change from iteration to iteration) and the train classifier(s) component 316 can output one or more initial classifiers.

A convolutional layer may include multiple filters (also referred to as kernels or activation functions). A filter inputs a convolutional window, for example, of received/measured clinical data associated with a patient, applies weights to clinical data type (e.g., urine output value(s), hydration fluid rate(s), diuretic infusion rate(s), heart rate(s), and/or other data described herein) of the convolutional window, and outputs an activation value for that convolutional window. For example, if the clinical data is times-series data collected over a period of 12 hours, the convolutional window may be 1 hour of the 12 hour period. The filter may apply a different weight to each of the clinical data in a given convolutional window to generate the activation value also referred to as a feature value. The convolutional layer may include, for each filter, a node (also referred to a neuron) for each clinical data type of the received/measured clinical data, assuming a stride of less than the convolutional window (e.g., less than 1 hour, such as 1 second, 1 minute, 5 minutes, 10 minutes, etc.) with appropriate padding. Each node outputs a feature value based on a set of weights for the filter that are learned by the optimizer of the model system 300 by adjusting the weights after each iteration

The ReLU layer may have a node for each node of the convolutional layer that generates a feature value. The generated feature values form a ReLU feature map. The ReLU layer applies a filter to each feature value of a convolutional feature map to generate feature values for a ReLU feature map. For example, a filter such as max (0, activation value) may be used to ensure that the feature values of the ReLU feature map are not negative.

The pooling layer may be used to reduce the size of the ReLU feature map by downsampling the ReLU feature map to form a pooling feature map. The pooling layer includes a pooling function that inputs a group of feature values of the ReLU feature map and outputs a feature value.

The FC layer includes some number of nodes that are each connected to every feature value of the pooling feature maps. Each node has a filter with its own set of weights.

The model system 300 further includes a build or train model component 310 and an apply model component 320. The build model component 310 may include a training data component 312, a data pre-processing component 314, and a train classifier(s) component 316. The apply model component 320 may include a patient data component 322, an apply classifier(s) component 324, and an output component 326. The build model component 310 and the apply model component may also share the classifier(s) 302 and/or the weight(s) 304 described in further detail above.

The training data component 312 can be configured to acquire, receive, and/or store clinical data associated with fluid therapy treatment for a plurality of patients, such as therapy duration, analyte concentration(s), urine output rate(s), diuretic delivery rate(s), hydration fluid delivery rate(s), patient outcome(s), any of the inputs and/or data described herein, and/or other suitable clinical data. The training data in the training data component 312 can be acquired/received from medical records, electronic medical records, data generated during fluid therapy treatment of past patient, and/or other suitable training data sources. The data pre-processing component 314 can be configured to pre-process or “clean” all or a portion of the data from the training data component 312. For example, the data pre-processing component 314 can be configured to remove or down-weight clinical data associated with a “bad ramp,” which may be determined based on an amount of time during which diuretic infusion was interrupted being greater than 10%, 20%, 30%, 40%, or 50% of the total time of the ramp. The train classifier(s) component 316 can generate one or more of the weights 304, and can iteratively update individual ones of the weights 304 and/or generate additional parameters as new training data is acquired, as described herein.

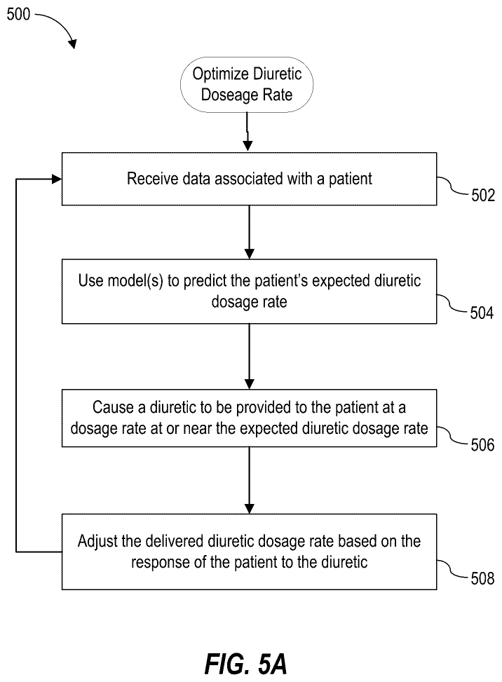

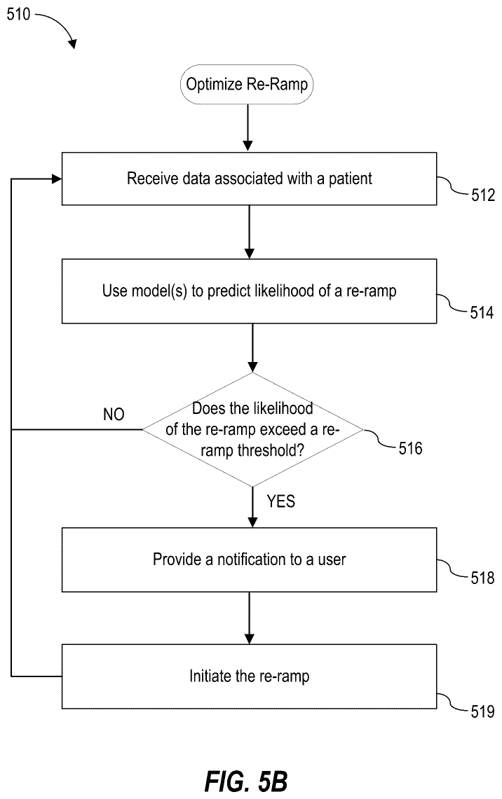

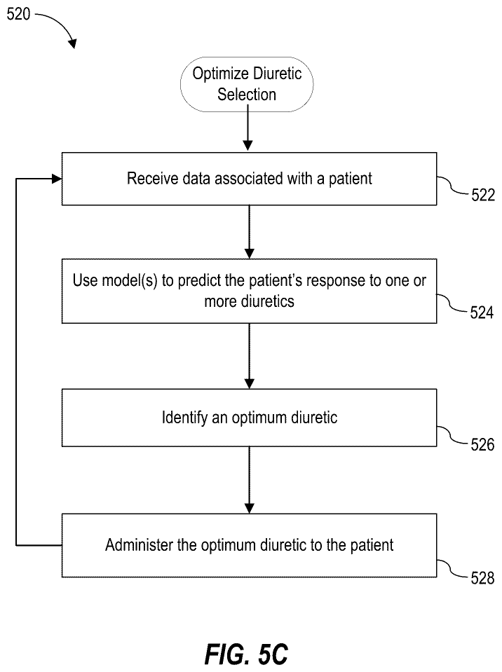

The patient data component 322 is configured to acquire, receive, and/or store patient data associated with the fluid therapy of a given patient, and can include any of the data types described herein (e.g., with reference to the training data component 312). The apply classifier(s) component 324 can apply the classifier(s) 302 to the patient data from the patient data component 322. In applying the classifier(s) 302, the apply classifier(s) component 324 can access one or more of the weights 304. The output component 326 can provide one or more outputs and/or perform one or more actions based at least partially on one or more results from the application of the classifier(s) 302 to the patient data in the patient data component 322 by the apply classifier(s) component 326. In at least some embodiments, the output component 326 transfers and/or stores a copy of the patient data from the patient data component 322 to the training data component 312, for example, to update the training data. In these and other embodiments, the output component 326 can provide one or more of the outputs and/or perform one or more of the actions described in detail with reference to FIGS. 5A-14.

The computing devices on which the model system 300 may be implemented may include a central processing unit and memory and may include input devices (e.g., keyboard and pointing devices), output devices (e.g., display devices), and storage devices (e.g., disk drives). Computer readable media include computer-readable storage media and data transmission media. The computer-readable storage media include memory and other storage devices that may have been recorded upon or may be encoded with computer executable instructions or logic that implement the classification system. The data transmission media is media for transmitting data using signals or carrier waves (e.g., electromagnetism) via a wire or wireless connection. Various functions of the classification system may also be implemented on devices with components that use discrete logic or logic embedded as an application-specific integrated circuit. The model system 300 may be implemented on a computer system that is local to the system 100 (FIG. 1). Alternatively, one or more of the components may be implemented on a computer system that is remote from the system 100. In such an alternative, the data used by the various components (e.g., return signals and image frames) may be transmitted between the local computing system and remote computer system and between remote computing systems.

The model system 300 may be described in the general context of computer-executable instructions, such as program modules, executed by one or more computers or other devices. Generally, program modules include routines, programs, objects, components, data structures, and so on that perform particular tasks or implement particular abstract data types. Typically, the functionality of the program modules may be combined or distributed as desired in various embodiments.

FIG. 3B is a block diagram that illustrates some of the components typically incorporated in at least some of the computer systems and other devices on which the disclosed systems 100, 300 can operate. In various embodiments, these computer systems and other devices 340 can include server computer systems, desktop computer systems, laptop computer systems, netbooks, mobile phones, personal digital assistants, televisions, cameras, automobile computers, electronic media players, etc. In various embodiments, the computer systems and devices include zero or more of each of the following: a central processing unit (CPU) 341 (such as the controller 140 of FIG. 1) for executing computer programs; a computer memory 342 for storing programs and data while they are being used, including the facility and associated data, an operating system including a kernel, and device drivers; a persistent storage device 343, such as a hard drive or flash drive for persistently storing programs and data; computer-readable media drives 344 that are tangible storage means that do not include a transitory, propagating signal, such as a floppy, CD-ROM, or DVD drive, for reading programs and data stored on a computer-readable medium; and a network connection 345 for connecting the computer system to other computer systems to send and/or receive data, such as via the Internet or another network and its networking hardware, such as switches, routers, repeaters, electrical cables and optical fibers, light emitters and receivers, radio transmitters and receivers, and the like. While computer systems configured as described above are typically used to support the operation of the facility, those skilled in the art will appreciate that the facility may be implemented using devices of various types and configurations, and having various components.

FIG. 3C is a system diagram illustrating an example of a computing environment in which the disclosed systems operate in some embodiments. In some embodiments, environment 350 includes one or more client computing devices 352A-D, examples of which can host various aspects of the disclosed systems 100, 300. Client computing devices 352 operate in a networked environment using logical connections through network 358 to one or more remote computers, such as a server computing device.

In some embodiments, server 354 is an edge server which receives client requests and coordinates fulfillment of those requests through other servers, such as servers 356A-C. In some embodiments, server computing devices 354 and 356a-c comprise computing systems, such as one or both of the systems 300, 340. Though each server computing device 354 and 356a-c is displayed logically as a single server, server computing devices can each be a distributed computing environment encompassing multiple computing devices located at the same or at geographically disparate physical locations. In some embodiments, each server 356 corresponds to a group of servers.

Client computing devices 352 and server computing devices 354 and 356a-c can each act as a server or client to other server or client devices. In some embodiments, servers (354, 356a-c) connect to a corresponding database (355, 357a-c). As discussed above, each server 354, 356a-c can correspond to a group of servers, and each of these servers can share a database or can have its own database. Databases 355 and 357 warehouse (e.g., store) information such as clinical data, patient data, model parameters, and so on. Though databases 355 and 357 are displayed logically as single units, databases 355 and 357 can each be a distributed computing environment encompassing multiple computing devices, can be located within their corresponding server, or can be located at the same or at geographically disparate physical locations.

Network 358 can be a local area network (LAN) or a wide area network (WAN), but can also be other wired or wireless networks. In some embodiments, network 358 is the Internet or some other public or private network. Client computing devices 352 are connected to network 358 through a network interface, such as by wired or wireless communication. While the connections between server 354 and servers 356a-c are shown as separate connections, these connections can be any kind of local, wide area, wired, or wireless network, including network 358 or a separate public or private network.

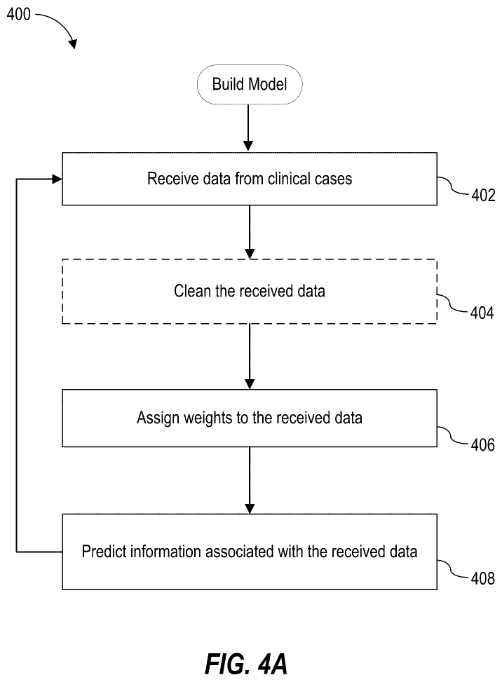

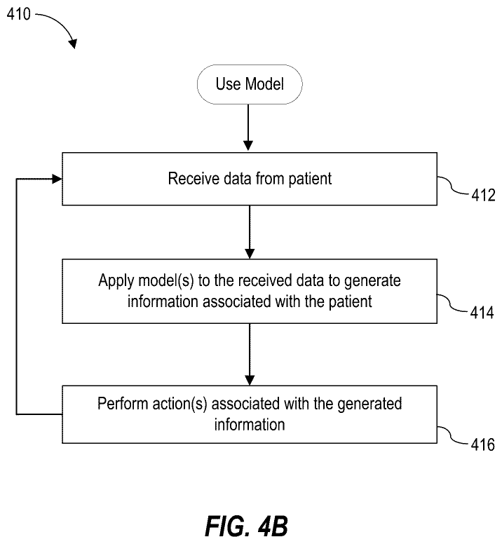

FIG. 4A is a block diagram illustrating a method 400 of building one or more models in accordance with embodiments of the present technology. The method 400 is illustrated as a series of process portions, blocks, or blocks 402-408, which can be performed by a model-building system, such as the model system 300 and/or the build model component 310 of FIG. 3A. Generally, the method 400 can be used to build any of the models described in further detail with reference to FIGS. 5A-14.

The method 400 begins at block 402 by receiving data from one or more clinical cases. The received data can be associated with one or more patients, e.g., that have or are currently undergoing fluid therapy treatment as described herein (e.g., with reference to FIGS. 1 and 2). For example, the received data can include values and/or identifiers associated with one or more of the following inputs and/or measurements: bioimpedance spectrometry, body temperature, continuous diuretic infusion rate, creatinine level, diastolic blood pressure, urine rate profile (e.g., slope of urine output curve), estimated excess fluid volume, GFR, heart rate, heart rate variability, hematocrit/hemoglobin level, hemodynamic measurements (e.g., from a catheter, an implant such as CardioMEMS, and the like), home diuretic dosage, hypotension, injected markers of hemoconcentration and/or GFR, IVC diameter, lung congestion (e.g., chest x-ray, ReDS), O₂ saturation, patient real time weight, patient therapy start weight, peak urine rate, prior need for therapy escalation (e.g., thiazide, high % saline matching, etc.), prior response to fluid therapy, respiration rate, serum analyte concentration(s), serum sodium level, signs and symptoms, systolic blood pressure, time to peak urine output, total urine volume, one or more urine analyte values (e.g., obtained via lab testing and/or sensor data), urine conductivity, urine diuretic concentration, urine output rate, and/or urine sodium level. Additionally or alternatively, the received data can include values and/or identifiers associated with any other inputs and/or measurements described herein and/or other suitable inputs and/or measurements. In some embodiments, block 402 includes receiving the data from a clinical or training data store, such as the training data component 312 of FIG. 3A.

At block 404, the method 400 can continue by pre-processing or cleaning all or a portion of the received data (block 402). In some embodiments, pre-processing or cleaning the received data can include removing or down-weighting clinical data associated with a “bad ramp,” as described herein (e.g., with reference to FIG. 3A). Additionally or alternatively, pre-processing or cleaning the received data can include removing or down-weighing outlier data, such as clinical data that differs (e.g., by more than a standard deviation) from one or more of the other clinical data and/or that include only a portion of the data associated with a given treatment period. In some embodiments, pre-processing or cleaning the received data includes normalizing the received data to remove outliers.

At block 406, the method 400 can continue by assigning weights to all or a subset of the received data (block 402), such as all or a subset of the cleaned data (block 404). Assigning the weights can include training one or more classifiers by applying the classifiers to training data to generate the weights, and assign each of the weights to a corresponding type of data (e.g., a first weight to urine output rate data, a second weight to GFR data, a third weight to estimated excess fluid volume, etc.). For example, block 406 can include training one or more classifier(s) 302 of FIG. 3A, by applying the classifiers to the training data stored within the training data component 312 of FIG. 3A to generate the weights(s) 304 of FIG. 3A.

At block 408, the method 400 can continue by predicting information associated with the received data (block 402). The predicted information can be based on an iterative training process for updating the weights and/or otherwise improving the accuracy of the model. For example, the predicted information can be compared against training data with known patient outcomes. In some embodiments, the predicted information and/or the patient outcomes can include one or more of the following: one or more diuretic ramp parameters (e.g., starting rate, curve shape, ending rate, ramp duration, time to initial urine response, slope of the urine response, and the like), a decision to re-ramp, a selection of and/or change to the diuretic, an adjustment to continuous diuretic infusion rate, an adjustment to the hydration fluid or saline infusion rate, an adjustment to the saline NaCl concentration, an adjustment to the hydration fluid composition (e.g., by adding other element(s)), a secondary diuretic type for delivery to the patient, an adjustment to a dosage of the secondary diuretic, an adjustment to the hydration fluid infusion rate, a decision to start/stop/continue/escalate therapy, a decision/recommendation to monitor the patient more closely following treatment, the selection of a home diuretic dose, a decision to inform a clinician of results of similar cases, an adjustment to the diuretic infusion rate, a decision to warn a user of a risk to the patient, a prediction of the time remaining until the hydration fluid sources should be replenished, and/or a decision to warn the user of a time to the hydration fluid source being empty. Additionally or alternatively, the predicted information and/or the patient outcomes can include other predicted information and/or patient outcomes described herein, and/or other suitable predicted information and/or patient outcomes. Based at least partially on a difference between the known patient outcomes and the predicted information, the method 400 may return to block 402 and repeat one or more of the blocks 402-408. For example, the method 400 can use data associated with actual user responses/actions to patient conditions and/or the patient's subsequent response to the user responses/actions, to further improve the accuracy of the prediction in block 408.

FIG. 4B is a block diagram illustrating a method 410 of using one or more models, such as the model(s) described with reference to FIGS. 3A-4A. The method 410 is illustrated as a series of steps, acts, processes, process portions, and/or blocks 412-416. At least some of the blocks 412-416 can be performed by a model application system, such as the model system 300 and/or the apply model component 320 of FIG. 3A. Generally, the method 410 can be used to determine any of the information and/or perform one or more of the actions described herein (e.g., with reference to FIGS. 5A-14).

The method 410 begins at block 412 by receiving data from a patient, which may be undergoing or planning to receive fluid therapy. The received data can include the data and/or data types described with reference to the received clinical data in block 402 of FIG. 4A. The data can be received from the system 100 of FIG. 1 (e.g., via the controller 140), one or more sensors (e.g., the sensors 114 of FIG. 1, external sensors, internal sensors, implantable sensors, wearable sensors, and/or the like), electronic medical records (EMR), lab test results, direct input (e.g., via a physician or user), and/or other suitable data sources.

At block 414, the method can continue by applying one or more models to the received data (block 412) to generate information associated with the patient. The models can include any model(s) described with reference to FIGS. 3A-4A, or another suitable model. The generated information can include and/or be associated with the patient outcomes and/or predicted information described with reference to block 408 in FIG. 4A. The application of the model(s) to the received data is described in further detail with reference to FIGS. 5A-14.

At block 416, the method 410 can continue by performing one or more actions associated with the generated information (block 414). For example, the one or more actions can include at least one of: recommending and/or selecting one or more diuretic ramp parameters (e.g., starting rate, curve shape, ending rate, and the like), a recommendation and/or decision to initiate a re-ramp, selecting and/or changing diuretic type, adjusting the continuous diuretic infusion rate, adjusting the saline infusion rate, adjusting the saline NaCl concentration, adjusting the saline composition (e.g., by adding other elements), delivering a secondary diuretic type to the patient (e.g., in addition to or in lieu of a first diuretic type), adjusting the secondary diuretic dose, adjusting the saline infusion rate, a recommendation and/or decision to stop or continue therapy, a recommendation and/or decision to follow the patient more closely after cessation of treatment, a recommendation and/or selection of home diuretic dose, informing the clinician of results of similar cases, adjusting the diuretic infusion rate, adjusting the hydration fluid infusion rate, slowing or stopping therapy, warning the user of a patient risk, warning the user of the time to the source being empty, a recommendation and/or decision regarding whether to start and/or resume therapy, and/or a recommendation and/or decision regarding when to stop therapy. The actions performed by the model(s) are described in further detail with reference to FIGS. 5A-14. Data associated with the actions and/or user responses to the actions can be used to further improve the accuracy of the method 400 and/or the method 410. For example, if the action includes a recommendation to continue therapy, but a user provides a response to stop therapy, this and other data associated with the recommendation, the user's response, and/or the patient's response (e.g., patient condition before and/or after the user response) can be provided to the models 400, 410 to improve their operation.

### III. Methods for Improving Fluid Therapy Treatment Systems and Devices

FIGS. 5A-14 are flowcharts illustrating respective methods 500, 510, 520, 600, 610, 700, 800, 810, 820, 830, 900, 1000, 1100, 1200, 1300, 1400 (“methods 500-1400”) for treating a patient in accordance with embodiments of the present technology. One or more of the methods 500-1400 can be used to treat the patient for fluid overload by removing fluid from the patient to produce a negative fluid balance (net fluid loss). The methods 500-1400 are described herein as a series of process portions, steps, or blocks, individual ones of which can be performed by any embodiment of the systems and devices described herein, such as the system 100 of FIG. 1. In some embodiments, at least some blocks of one or more of the methods 500-1400 are performed by a system or device including one or more processors and a memory storing instructions that, when executed by the one or more processors, cause the system or device to perform one or more of the blocks described herein. For example, one or more of the methods 500-1400 can be performed by the controller 140 of the system 100 of FIG. 1, the model system 300 of FIG. 3A, the CPU 341 of FIG. 3B, or another suitable computing device/system. All or a portion of one or more of the methods described herein can be performed on a single processing device, on multiple processing devices, and/or in a networked or cloud computing environment. Additionally, or alternatively, at least some blocks of one or more of the methods 500-1400 can be performed by a model, such as any of the models described with reference to FIGS. 3A-4A. Optionally, some or all of the blocks of one or more of the methods can performed automatically or semi-automatically, with little or no human intervention.

The method 500 of FIG. 5A is generally associated with optimizing a dosage rate of diuretic provided to a patient during fluid therapy. An optimized diuretic dosage rate is expected to improve the patient specificity and/or granularity of diuretic dosing, and/or reduce or prevent over-administration of diuretic. Referring to FIG. 5A, the method 500 can begin at block 502 by receiving data associated with a patient. Block 502 can be generally similar or identical to block 412 of FIG. 4B. The received data can be associated with the patient's baseline condition, and/or the patient's condition after obtaining the urine output rate (e.g., block 202 in FIG. 2). In some embodiments, the received data can include one or more of: a urine output rate, a total urine volume, a peak urine rate, a urine rate profile (e.g., a slope of urine output rate curve), a time to peak urine output, a downward slope of urine output curve, a home diuretic dosage, a GFR, creatine levels, prior response to fluid therapy, prior need for therapy escalation (e.g., thiazide, high % saline matching, etc.), bioimpedance spectrometry data, a heart rate, a heart rate variability, a respiration rate, a urine diuretic concentration, urine sodium data, serum sodium data, urine conductivity data, urine analyte data, serum analyte data, a systolic blood pressure, a diastolic blood pressure, patient therapy start weight, patient real time weight, an estimated excess fluid volume, hematocrit/hemoglobin, an O₂ saturation, injected markers of hemoconcentration/GFR, lung congestion (e.g., chest x-ray, ReDS), hemodynamic measurements, body temp, inferior vena cava (IVC) diameter, signs and symptoms, and/or other data described herein.

At block 504, the method 500 can continue by using one or more models to predict an expected diuretic dosage rate of the patient. The expected diuretic dosage rate can be predicted based at least partially on the data received in block 502. For example, the model(s) can be configured/trained to compare the data received in block 502 with historical/training data from one or more other patients, and identify the diuretic dosage rate for patients having generally similar or identical treatment profiles.

At block 506, the method 500 can continue by causing a diuretic to be provided to the patient at a dosage rate at or near the expected diuretic dosage rate. Causing the diuretic to be provided can include providing instructions, e.g., via non-transitory computer-readable media, to the pump or device fluidly coupled to the diuretic, and/or to nurse, physician, or other user to set the diuretic infusion rate to be at or near the expected diuretic dosage rate.

At block 508, the method 500 can continue by adjusting the delivered diuretic dosage rate based on the response of the patient to the diuretic. For example, based on the difference between the patient's actual urine output and desired urine output, the expected diuretic dosage rate can be increased or decreased to at least partially reduce the difference between the actual and desired urine outputs. In some embodiments, block 508 includes “ramping” or increasing the expected diuretic dosage rate according to a desired function or delivery profile, as described herein. For example, the delivered diuretic dosage rate can be ramped in a manner such that a subsequent dosage rate is a predetermined percentage (e.g., at least 5%, 10%, 15%, 25%, 50%, 100%, etc.) above the expected diuretic dosage rate. If a predetermined amount of time (e.g., at least 10 minutes, 30 minutes, 1 hour, 2 hours, etc.) has passed without the patient achieving the desired urine output, the delivered diuretic dosage rate can be increased to a maximum diuretic dosage rate (e.g., at least 150%, 200%, etc.) of the expected diuretic dosage rage.

In some embodiments, the method 500 can return to block 502 and/or repeat one or more of the blocks 502-508, for example, in response to receiving new/updated data associated with the patient. As such, the method 500 can be iterative to continuously improve or optimize the delivered diuretic dosage rate of the patient.

The method 510 of FIG. 5B is generally associated with predicting whether the patient's initial diuretic dosage rate will be increased or “re-ramped” as the fluid therapy progresses, and recommending and/or automatically executing the increase/re-ramp based on the patient's condition. Predicting fluid therapy re-ramps reduce or prevent periods during the fluid therapy where the patient's urine output is interrupted or falls below a minimum level. The method 510 can begin at block 512 by receiving data associated with the patient. Block 512 can be generally similar or identical to block 502 of FIG. 5A. The received data can be associated with the patient's baseline condition, and/or the patient's condition after obtaining the urine output rate (e.g., block 202 in FIG. 2). In some embodiments, the received data can include one or more of: a urine output rate, a total urine volume, a peak urine rate, a slope of urine output rate curve, a time to peak urine output, a downward slope of urine output curve, a home diuretic dosage, a GFR, creatine levels, prior response to fluid therapy, prior need for therapy escalation (e.g., thiazide, high % saline matching, etc.), bioimpedance spectrometry, heart rate, heart rate variability, respiration rate, urine diuretic concentration, urine sodium, serum sodium, urine conductivity, urine analytes, serum analytes, systolic blood pressure, diastolic blood pressure, hypotension, patient therapy start weight, patient real time weight, estimated excess fluid volume, hematocrit/hemoglobin, O₂ sat, injected markers of hemoconcentration/GFR, lung congestion (e.g., chest x-ray, ReDS), hemodynamic measurements from catheter, hemodynamic measurements from implants like CardioMEMS, body temp, IVC diameter, signs and symptoms (difficult to measure in real time), and/or any of the other data described herein.

At block 514, the method 510 can continue by using one or more models to predict a likelihood of a re-ramp. The likelihood of the re-ramp can be predicted based at least partially on the data received in block 512. For example, the model(s) can be configured/trained to compare the data received in block 512 with historical/training data from one or more other patients, and identify instance(s) of fluid therapy re-ramps (e.g., re-ramp occurrence rates) for others patients having generally similar or identical treatment profiles.

At block 516, the method 510 can continue by determining whether the predicted likelihood of the re-ramp (block 514) exceeds a re-ramp threshold. If the predicted likelihood of the re-ramp exceeds the re-ramp threshold (e.g., block 516, “YES”), the method 510 can continue to block 518. If the predicted likelihood of the re-ramp is less than or equal to the re-ramp threshold (e.g., block 516, “NO”), then the method 500 can return to block 512 and/or repeat one or more of the blocks 512-516. The re-ramp threshold can be a percent likelihood between about 10% and about 99%, such as 80%, or another suitable percent likelihood therebetween.

At block 518, the method 510 can continue by providing a notification to a user (e.g., a clinician, a practitioner, and the like). The notification can include a visual notification, a text-based notification, a tactile notification, an audible notification, and/or another suitable notification. In at least some embodiments, for examples, block 518 includes displaying a message to the user via the display 150 of the system 100 of FIG. 1 indicating that the predicted likelihood of the re-ramp exceeds the re-ramp threshold and/or that the patient will likely require a re-ramp. Alternately, block 518 may be skipped and the method 510 can proceed to block 519.

At block 519, the method 510 can continue by initiating the re-ramp, for example, by increasing the dosage rate of the diuretic administered to the patient. In some embodiments, the re-ramp can be initiated manually (e.g., by a user), automatically (e.g., by the system 100 of FIG. 1), or semi-automatically, as described in detail herein.

The method 520 of FIG. 5C is generally directed to selecting an optimal diuretic to deliver to the patient. In some aspects, delivering the optimal diuretic is expected to increase (e.g., maximize) the patient's urine output to the fluid therapy and/or further reduce the likelihood that the patient will experience adverse events in response to the diuretic. The method 520 can begin at block 522 by receiving data associated with the patient. Block 522 can be generally similar or identical to block 512. The received data can be associated with the patient's baseline condition, and/or the patient's condition after obtaining the urine output rate (e.g., block 202 in FIG. 2). In some embodiments, the received data can include one or more of: a urine output rate, a total urine volume, a peak urine rate, a slope of urine output rate curve, a time to peak urine output, a downward slope of urine output curve, a home diuretic dosage, GFR, creatine levels, prior response to fluid therapy, prior need for therapy escalation (e.g., thiazide, high % hydration fluid matching, etc.), bioimpedance spectrometry, heart rate, heart rate variability, respiration rate, urine diuretic concentration, urine sodium, serum sodium, urine conductivity, urine analytes, serum analytes, systolic blood pressure, diastolic blood pressure, hypotension, patient therapy start weight, patient real time weight, estimated excess fluid volume, hematocrit/hemoglobin, O₂ sat, injected markers of hemoconcentration/GFR, lung congestion (e.g., chest x-ray, ReDS), hemodynamic measurements from catheter, hemodynamic measurements from implants like CardioMEMS, body temp, IVC diameter, signs and symptoms (difficult to measure in real time), and/or any of the other data described herein.

At block 524, the method 520 can continue by using one or more models to predict the patient's response to one or more diuretics. In some embodiments, block 524 can include predicting the patient's expected urine output, expected diuretic dosage rate (e.g., method 500 of FIG. 5A), and/or the likelihood of one or more adverse events (e.g., over-diuresis, dehydration, and/or intravascular depletion).

At block 526, the method 520 can continue by identifying an optimum diuretic. The optimum diuretic can be associated with an increased expected urine output, a decreased expected diuretic dosage rate, and/or a reduced likelihood of one or more adverse events relative to one or more other diuretics. For example, of all diuretics evaluated in block 514, the optimum diuretic can be associated with the highest expected urine output, the lowest expected diuretic dosage rate, and/or the lowest likelihood of one or more adverse events. Additionally, or alternatively, block 526 can include presenting the predicted patient responses (block 524) to the user so that the user can identify the optimum diuretic.

At block 528, the method 520 can continue by administering the optimum diuretic (block 526) to the patient. In at least some embodiments, for example, block 528 can include causing the system 100 to automatically deliver the optimum diuretic to the patient and/or prompting a user to administer the optimum diuretic to the patient.

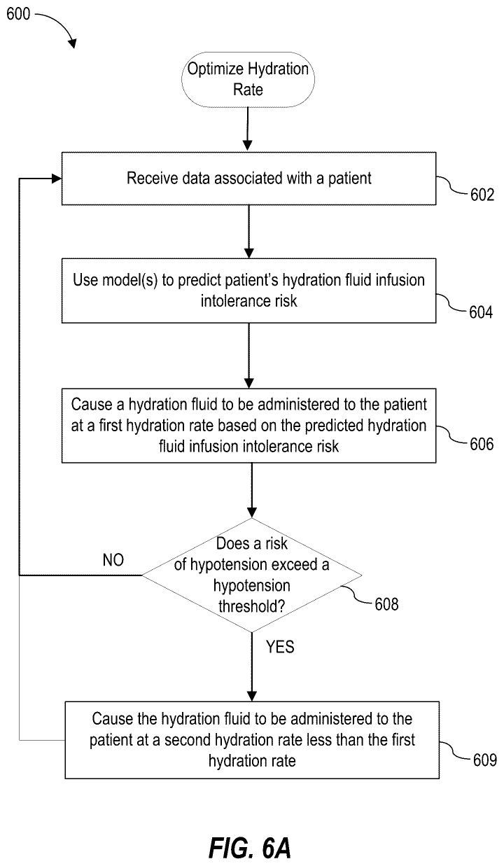

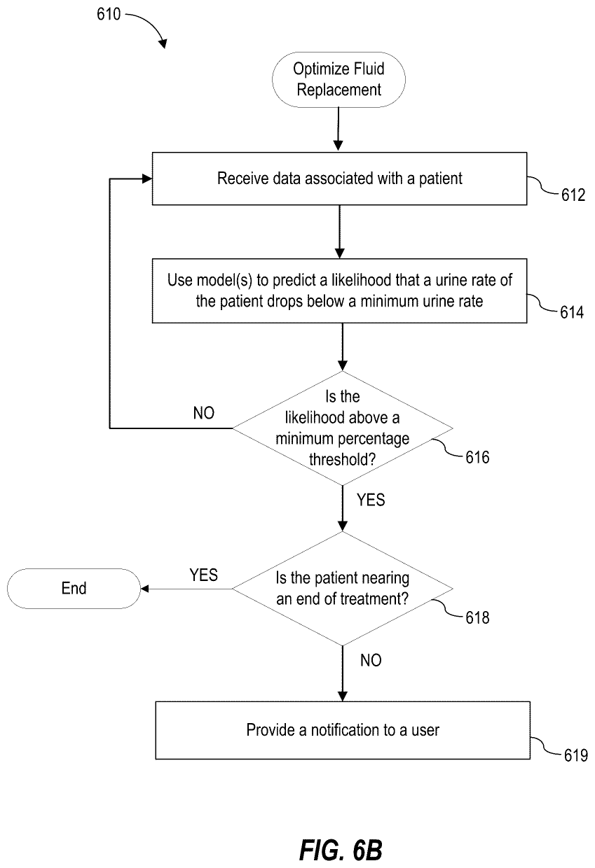

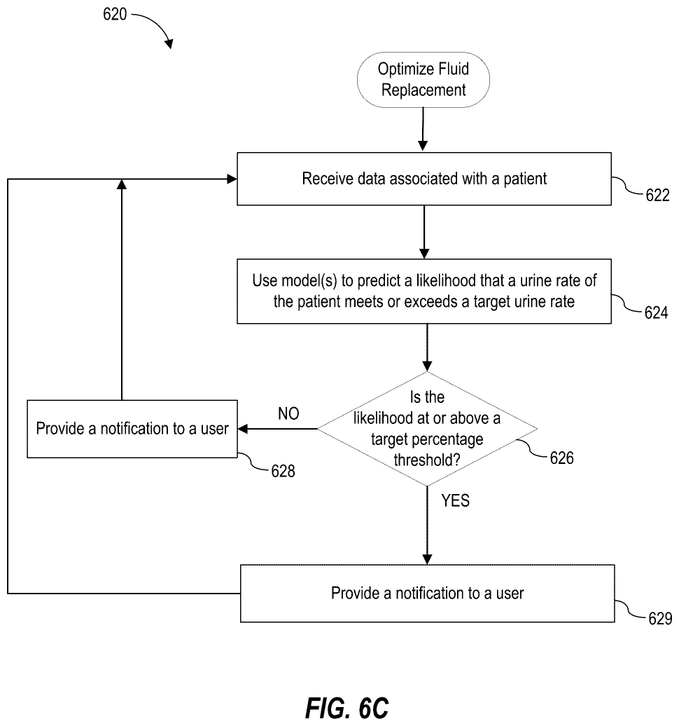

FIGS. 6A-6C illustrate respective methods 600, 610, 620 associated with hydration fluid delivery during fluid therapy, in accordance with embodiments of the present technology. One or more of the methods 600, 610, 620 can be used to optimize the initial delivery of hydration fluid, and/or optimize the patient's urine output. Each of the methods 600, 610, 620 can include one or more models trained using clinical/historical patient data, as described with reference to FIG. 4A. For example, one or more of the models can be trained based on patient data including one or more of the following: urine output (e.g., rate, change in urine output rate, volume, and the like), leaky aortic or mitral heart valve or heart valve replacement, valvular heart conditions, ejection fraction, pulmonary wedge pressure, systolic and/or diastolic blood pressure, estimated excess fluid, GFR, biomarker(s), hydration fluid volume infused, heart rate, respiration rate, and/or other suitable data described herein. Generally, based on the data associated with a patient, one or more of the models can be configured to adjust the patient's hydration rate, adjust the concentration of one or more compounds (e.g., NaCl) in the hydration fluid, and/or otherwise adjust the hydration fluid composition (e.g., by adding and/or removing one or more other compounds).

The method 600 of FIG. 6A is generally directed to optimizing or improving the initial delivery of hydration fluid, such as by reducing one or more risks associated with the initial delivery of the hydration fluid. The method 600 can begin at block 602 by receiving data associated with a patient. Block 602 can be generally similar or identical to block 412 of FIG. 4B. The received data can be associated with the patient's baseline condition, and/or the patient's condition after obtaining the urine output rate (e.g., block 202 in FIG. 2). In some embodiments, the received data can include one or more of: urine output rate, total urine volume, peak urine rate, slope of urine output rate curve, time to peak urine output, downward slope of urine output curve, urine sodium, serum, sodium, GFR, creatinine, urine conductivity, urine analytes, serum analytes, systolic blood pressure, diastolic blood pressure, patient therapy start weight, patient real time weight, estimated excess fluid volume, and/or any of the other data described herein.

At block 604, the method 600 can continue by using one or more models to predict the patient's hydration fluid infusion intolerance risk. The hydration fluid intolerance risk can include the likelihood that a hydration rate matching the patient's urine output (e.g., 100% of the urine output rate) worsens and/or otherwise increases the patient's risk of experiencing one or more adverse events, such as pulmonary edema. The model(s) can predict the patient's hydration fluid intolerance risk based at least partially on the data received in block 602. For example, the model(s) can be configured/trained to compare the data received in block 602 with historical/training data from one or more other patients, and identify instance(s) of hydration fluid intolerance for others patients having generally similar or identical treatment profiles.

At block 606, the method 600 can continue by causing a hydration fluid to be administered to the patient at a first hydration rate based at least partially on the predicted hydration fluid infusion intolerance risk (block 604). The first hydration rate can be less then, equal to, or greater than the patient's urine output. For example, if the predicted hydration fluid infusion intolerance risk is high (e.g., greater than 70%), the first hydration rate can be low, such as a rate matching a first amount (e.g., the first 0-100 mL) of the patient's urine output. In at least some embodiments, if the predicted hydration fluid infusion intolerance risk is sufficiently high (e.g., greater than 80%), no hydration fluid may be delivered to the patient. If the predicted hydration fluid infusion intolerance risk is moderate (e.g., between about 30% and about 70%), the first hydration rate can be moderate, such as a rate matching up to a second amount (e.g., the first 100-349 mL) of the patient's urine output, or another value therebetween. The second amount can be greater than the first amount. If the predicted hydration fluid infusion intolerance risk is low (e.g., between about 0% and about 30%), the first hydration rate can be high, such as a rate matching at least a third amount (e.g., at least the first 350 mL, the first 500 mL, or another suitable volume) of the patient's urine output. The third amount can be greater than the first amount and/or second amount. In such embodiments, the hydration rate relative to the urine output rate can vary over time depending on the current urine output rate. For example, the hydration rate may 100% match the first 0-100 mL of the patient's urine output and then match a different percentage (e.g., 90%, 80%, 70%, etc.) of the next 100 mL of the patient's urine output.

At block 608, the method 600 can continue by determining whether, in response to administering the hydration fluid at the first hydration fluid rate, a risk of hypotension (or another adverse event associated with patient overhydration, such as acute kidney injury) exceeds a hypotension threshold. The hypotension threshold can be a likelihood greater than 25%, 50%, 75%, or any likelihood therebetween. The hypotension threshold can be determined by a user and/or by one or more of the models (block 604). If the risk of hypotension does not exceed the hypotension threshold (block 608, “NO”), the method 600 can return to block 602 and/or repeat one or more of the blocks 602-608. If the risk of hypotension exceeds the hypotension threshold (block 608, “YES”), the method 600 can continue to block 609.

At block 609, the method 600 can continue by causing the hydration fluid to be administered to the patient at a second hydration rate less than the first hydration rate. In some embodiments, the hydration fluid is administered at the second hydration rate only after the rate matching described in block 606 has been completed, such as after the patient's urine output has reached a predetermined level associated with the rate matching. The second hydration fluid rate can be determined by a user and/or by one or more of the models (block 604). In some embodiments, the second hydration fluid rate is expected to prevent, or at least reduce the likelihood of, the patient experiencing hypotension in response to delivery of the hydration fluid.

The method 610 of FIG. 6B is generally directed to optimizing the patient's urine output and/or fluid replacement. The method 610 can begin at block 612 by receiving data associated with a patient. Block 612 can be generally similar or identical to block 602 of FIG. 6A. The received data can be associated with the patient's baseline condition, and/or the patient's condition after obtaining the urine output rate (e.g., block 202 in FIG. 2). In some embodiments, the received data can include one or more of: urine output rate, total urine volume, peak urine rate, slope of urine output rate curve, time to peak urine output, downward slope of urine output curve, urine sodium, serum, sodium, GFR, creatinine, urine conductivity, urine analytes, serum analytes, systolic blood pressure, diastolic blood pressure, patient therapy start weight, patient real time weight, estimated excess fluid volume, and/or any of the other data described herein.

At block 614, the method 610 can continue by using one or more models to predict a likelihood that a urine rate of the patient falls below a minimum urine rate. The likelihood that the urine rate falls below the minimum urine rate can be based at least partially on the data received in block 612. For example, the model(s) can be configured/trained to compare the data received in block 612 with historical/training data from one or more other patients, and identify instance(s) where a urine rate for others patient(s) having generally similar or identical treatment profiles fell below a corresponding minimum urine rate.

The minimum urine rate can be between about 0 mL/hr and about 450 mL/hr, such as about 325 mL/hr, or another urine rate there between. In some embodiments, one or more of the models can be configured to predict the likelihood that the urine rate falls below the minimum urine rate within a time period. The time period can be between about 1 minute and about 12 hours, such as at least 2 hours, or another duration of time therebetween.

At block 616, the method 610 can continue by determining whether the predicted likelihood in block 614 is above a minimum percentage threshold. The minimum percentage threshold can be between about 50% and about 95%, such as at least 80%, or another likelihood therebetween. The minimum percentage threshold can be determined by a user and/or automatically determined by one or more of the models (block 614). If the likelihood is less than or equal to the minimum percentage threshold (block 616, “NO”), the method 610 can return to block 612 and/or repeat one or more of the blocks 612-616. If the likelihood is greater than the minimum urine rate threshold (block 616, “YES”), the method 610 can continue to block 618.

At block 618, the method 610 can continue by determining whether the patient is nearing an end of their fluid therapy treatment. In some embodiments, block 618 can include determining, via one or more of the models (block 614) a percent likelihood that the patient's fluid therapy will be stopped within the time period (block 614). If the patient is nearing the end of treatment (block 618, “YES”), then the method can end. If the patient is not nearing the end of treatment (block 618, “NO”), then the method 610 can continue to block 619.

At block 619, the method 610 can continue by providing a notification (e.g., an alert, a message, etc.) to a user. The notification can be associated with the results of block 616 and/or block 618. For example, the notification can be configured to alert a user that the likelihood of patient's the urine rate dropping below the minimum urine rate is above the minimum urine rate threshold and thus the user should consider increasing hydration fluid matching and/or that the patient is not nearing the end of their treatment.

The method 620 of FIG. 6C is generally directed to optimizing the patient's urine output and/or fluid replacement. The method 620 can begin at block 622 by receiving data associated with a patient. Block 622 can be generally similar or identical to block 602 of FIG. 6A and/or block 612 of FIG. 6B. The received data can be associated with the patient's baseline condition, and/or the patient's condition after obtaining the urine output rate (e.g., block 202 in FIG. 2). In some embodiments, the received data can include one or more of: urine output rate, total urine volume, peak urine rate, slope of urine output rate curve, time to peak urine output, downward slope of urine output curve, urine sodium, serum, sodium, GFR, creatinine, urine conductivity, urine analytes, serum analytes, systolic blood pressure, diastolic blood pressure, patient therapy start weight, patient real time weight, estimated excess fluid volume, and/or any of the other data described herein.

At block 624, the method 620 can continue by using one or more models to predict a likelihood that a urine rate of the patient meets or exceeds a target urine rate. The likelihood that the urine rate meets or exceeds the target urine rate can be based at least partially on the data received in block 622. For example, the model(s) can be configured/trained to compare the data received in block 622 with historical/training data from one or more other patients, and identify instance(s) where a urine rate for others patient(s) having generally similar or identical treatment profiles met or exceeded a corresponding target urine rate. In at least some embodiments, block 624 can be performed before the start of fluid therapy, and the predicted likelihood can represent the likelihood that the urine rate of the patient meets or exceeds the target urine rate once fluid therapy begins.

The target urine rate can be between about 0 mL/hr and about 600 mL/hr, such as at least 300 mL/hr, at least 325 mL/hr, at least 350 mL/hr, 400 mL/hr, 525 mL/hr, or another urine rate therebetween. In some embodiments, one or more of the models can be configured to predict the likelihood that the urine rate meets or exceeds the target urine rate within a time period. The time period can be between about 1 minute and about 12 hours, such as at least 2 hours, during the therapy period, or another duration of time therebetween.

At block 626, the method 620 can continue by determining whether the predicted likelihood in block 624 is at or above a target percentage threshold. The target percentage threshold can be between about 50% and about 95%, such as at least 80%, or another likelihood therebetween. The target percentage threshold can be determined by a user and/or automatically determined by one or more of the models (block 624). If the likelihood is less than or equal to the target percentage threshold (block 626, “NO”), the method 620 can continue to block 628 and/or repeat one or more of the blocks 622-626. If the likelihood is greater than the minimum urine rate threshold (block 626, “YES”), the method 620 can continue to block 629 and/or repeat one or more of the blocks 622-626.

At block 628, the method 620 can continue by providing a notification (e.g., an alert, a message, etc.) to a user. The notification can be associated with the results of block 626. For example, the notification can be configured to alert a user that the likelihood of patient's the urine rate meeting or exceeding the target urine rate is less than the target percentage threshold.

At block 629, the method 620 can continue by providing a notification (e.g., an alert, a message, etc.) to a user. The notification can be associated with the results of block 626. For example, the notification can be configured to alert a user that the likelihood of the patient's urine rate meeting or exceeding the target urine rate meets or exceeds the target percentage threshold.

Referring to block 628 and block 629 together, one or more aspects of the fluid therapy can be adjusted based on whether the predicted likelihood (block 624) is at or above the target percentage threshold, such as in response to one or both of the notifications in block 628 and block 629. For example, the predicted likelihood being below the target percentage threshold (block 628) can indicate that the patient is expected to be unresponsive or insensitive to diuretics and/or hydration fluid, and/or that the patient should receive an increased delivery rate of a diuretic (e.g., a higher initial or starting delivery rate) and/or increased hydration fluid matching. Thus, a higher delivery rate of a diuretic (e.g., a higher initial or starting delivery rate) can be administered to the patient to attempt to increase the likelihood that the patient's urine rate meets or exceeds the target urine rate. If, on the other hand, the predicted likelihood is at or above the target percentage threshold (block 629), this can indicate that the patient is generally expected to be responsive or sensitive to diuretics and/or hydration fluid, and a lower delivery rate of a diuretic (e.g., a lower initial or starting delivery rate) and/or reduced hydration fluid matching can be administered to the patient, without or substantially without affecting the likelihood that the urine rate meets or exceeds the target urine rate. Additionally, or alternatively, one or both of the notifications in block 628 and block 629 can include a predicted diuretic type, diuretic delivery rate, and/or an adjustment to a diuretic delivery rate that is expected to cause the urine rate of the patient to meet or exceed the target urine rate.

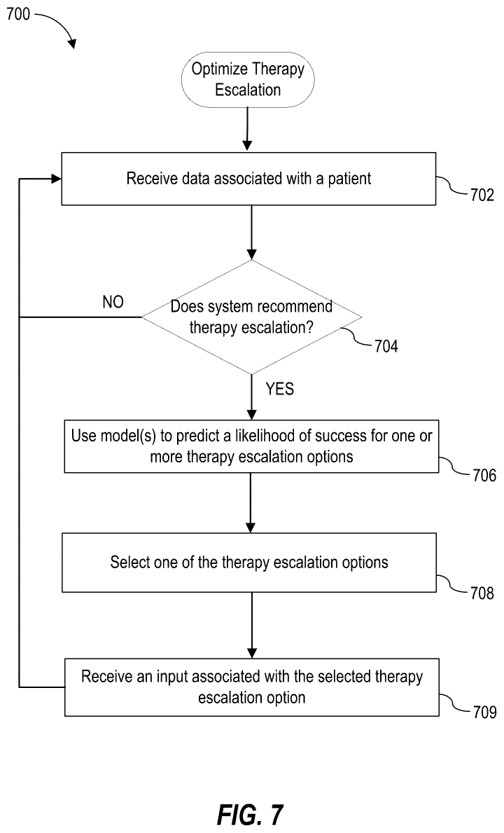

FIG. 7 illustrates a method 700 associated with escalating a patient's fluid therapy in accordance with embodiments of the present technology. In some embodiments, the method 700 can be used to optimize the time at which the patient therapy is escalated and/or predict whether the patient therapy will be escalated. The method 700 can include one or more models trained using clinical/historical patient data, as described with reference to FIG. 4A. For example, one or more of the models can be trained based on patient data include one or more of the following: urine output (e.g., current rate, total volume, peak rate, slope up in response to dose finding algorithm, time to peak, slope down, etc.), occurrence(s) of therapy escalation, type of therapy escalation (e.g., thiazide or temporary fluid matching), continuous diuretic infusion rate, home diuretic dosage, estimated excess fluid volume, GFR, and/or other suitable data described herein. Generally, based on the data associated with a patient, one or more of the models can be configured to recommend or administer an additional (e.g., second) diuretic type for delivery to the patient, adjust a dosage and/or delivery rate for the additional diuretic, adjust the hydration fluid rate, and/or perform other therapy escalations.

The method 700 can begin at block 702 by receiving data associated with a patient. Block 702 can be generally similar or identical to block 412 of FIG. 4B. The received data can be associated with the patient's baseline condition, and/or the patient's condition after obtaining the urine output rate (e.g., block 202 in FIG. 2). In some embodiments, the received data can include one or more of: a urine output rate, total urine volume, peak urine rate, slope of urine output rate curve, time to peak urine output, downward slope of urine output curve, continuous diuretic infusion rate, home diuretic dosage, prior response to reprieve therapy, estimated excess fluid volume, GFR, creatinine levels, and/or any of the other data described herein.

At block 704, the method 700 can continue by determining whether a fluid therapy system, such as the system 100 of FIG. 1, recommends escalating the patient's therapy, e.g., administering a second diuretic (e.g., thiazide), temporarily increased hydration fluid matching, administering additional loop diuretic, using continuous veno-venous hemofiltration, using ultrafiltration, and/or via a cardiac assist device, and/or an indication whether one or more of the therapy escalations is expected to improve the patient's condition. The recommendation can be based at least partially on the data received in block 702. For example, the model(s) can be configured/trained to compare the data received in block 702 with historical/training data from one or more other patients, and identify instance(s) and/or historical success rates of therapy escalations for others patients having generally similar or identical treatment profiles. If the fluid therapy system does not recommend escalating the patient's therapy (block 704, “NO”), the method 700 can return to block 702 and/or repeat one or more of the blocks 702-704. If the fluid therapy system does recommend escalating the patient's therapy (block 704, “YES”), then the method 700 can continue to block 706.

At block 706, the method 700 can continue by using one or more models to predict a likelihood of a successful patient response (e.g., improved patient outcome, increased urine output, reduced risk of an adverse event, reduced treatment duration, and the like) for one or more therapy escalation options. For example, block 706 can include predicting the likelihood of a successful patient response to the delivery of thiazide, temporary fluid matching, administration of additional loop diuretic, or another therapy escalation option.

At block 708, the method 700 can continue by selecting and/or recommending at least one of the therapy escalation options of block 706. In some embodiments, the selected therapy escalation option can be the therapy escalation option associated with the best (e.g., most successful) patient response (e.g., most improved outcome, highest urine output, lowest risk of an adverse event, lowest treatment duration, and the like). At least one of the selected therapy escalation options can be selected manually by a user and/or automatically recommended and/or selected by one or more of the models (block 706).

At block 709, the method 700 can optionally continue by receiving an input associated with the selected therapy option. For example, the input can be an input provided by the user corresponding to the selected therapy option, which can be logged by one or more of the models and used to further improve future predictions (block 706) and/or recommendations/selections (block 708) for therapy escalation. Additionally or alternatively, the input can include urine output data from the patient associated with the patient's response to the selected therapy escalation option, which can be logged by one or more of the models and user to further improve future predictions (block 706).

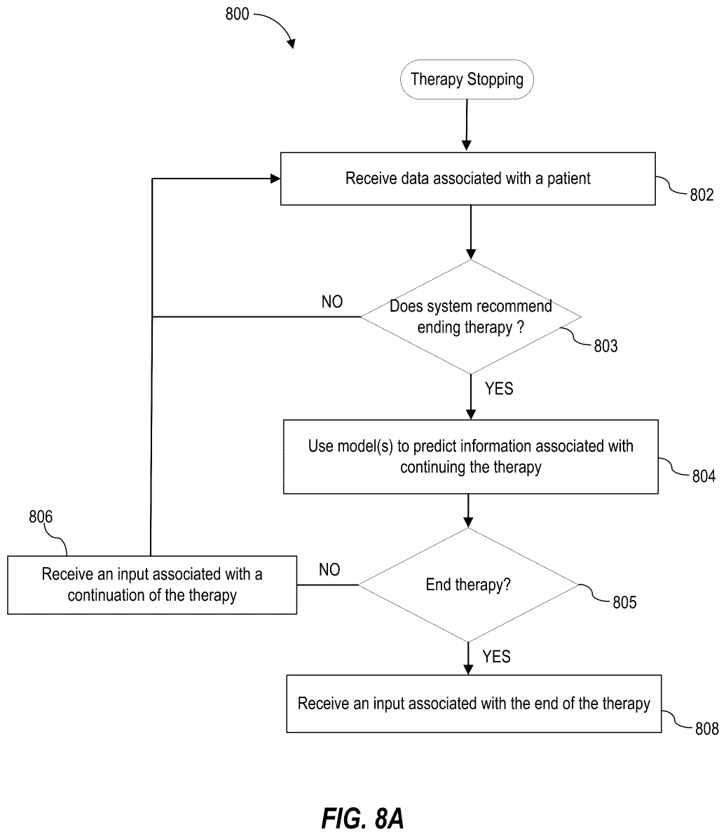

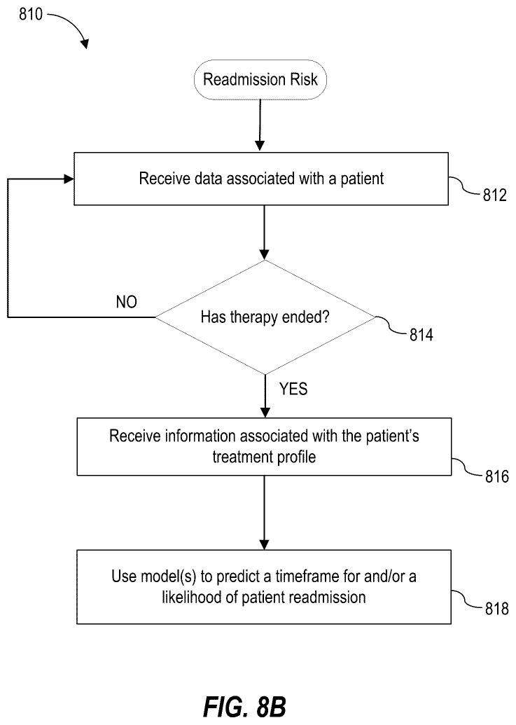

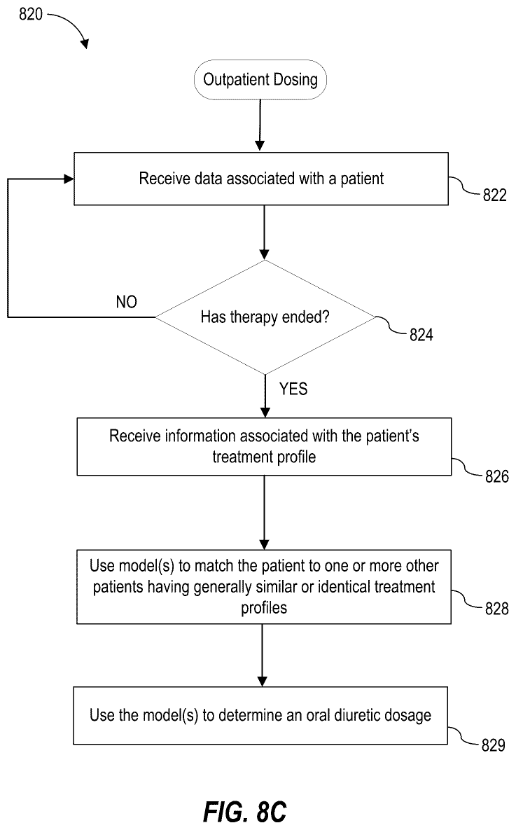

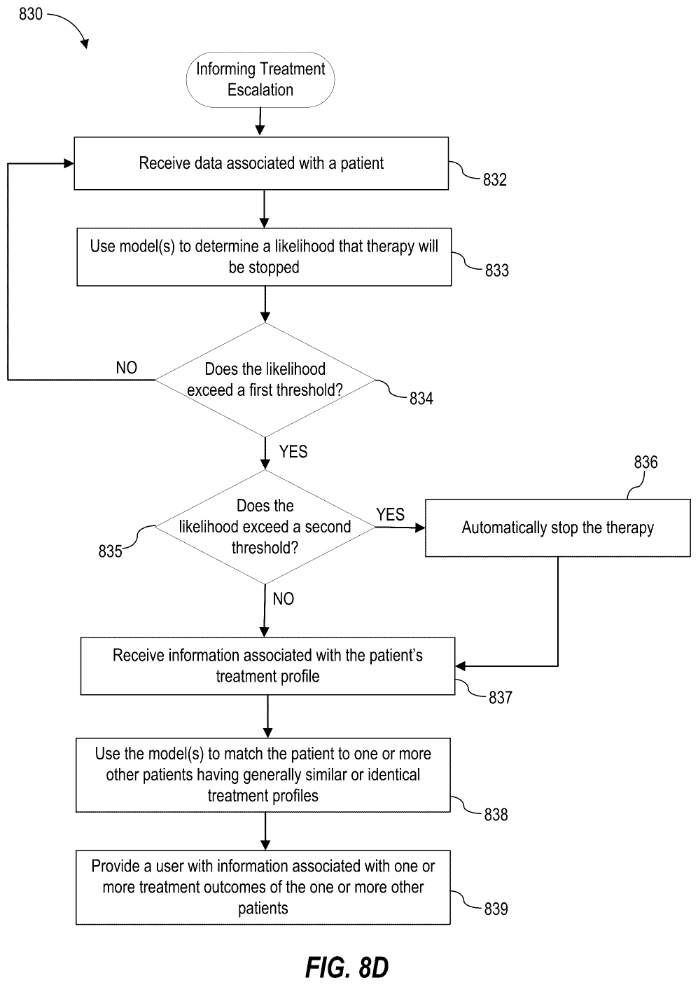

FIGS. 8A-8D illustrate respective methods 800, 810, 820, 830 associated with ending a patient's fluid therapy, in accordance with embodiments of the present technology. One or more of the methods 800, 810, 820, 830 can be used to support a user's decision to stop the patient's fluid therapy, automatically stop the patient's fluid therapy, determine the patient's readmission risk, determine information associated with oral diuretic dosing, determine information associated with patient post-treatment care in the event that the patient does not respond to the fluid therapy, estimate therapy duration, predict the occurrence of euvolemia post-treatment, or determine other suitable information associated with ending the patient's fluid therapy. Each of the methods 800, 810, 820, 830 can include one or more models trained using clinical/historical patient data, as described with reference to FIG. 4A. For example, one or more of the models can be trained based on patient data including one or more of the following: urine output (e.g., current rate, total volume, peak rate, slope up in response to DFA, time to peak, slope down, etc.), occurrence(s) of therapy conclusion recommendation and/or subsequent clinical decision to continue or conclude, continuous diuretic infusion rate, therapy duration, home diuretic dosage, estimated excess fluid volume, GFR, date of most recent prior admission, admission and discharge weight, admission and discharge vitals (e.g., heart rate, respiratory rate, blood pressure, and the like), date of subsequent re-admission, therapy escalation/thiazide use, oral dose prescribed at discharge, and/or other suitable data described herein. Generally, based on the data associated with a patient, one or more of the models can be configured to provide a recommendation and/or decide to stop or continue the patient's fluid therapy, provide a recommendation to follow the patient more closely after the end of the patient's fluid therapy, recommend and/or automatically determine a dosage of a diuretic for the patient to self-administer after the end of their fluid therapy, provide a clinician with treatment data associated with similar patients, and/or other suitable outputs associated with ending the patient's fluid therapy.

The method 800 of FIG. 8A is associated with automatically stopping and/or providing information to support a decision to stop a patient's fluid therapy, in accordance with embodiments of the present technology. The method 800 can begin at block 802 by receiving data associated with a patient. Block 802 can be generally similar or identical to block 412 of FIG. 4B. The received data can be associated with the patient's baseline condition, and/or the patient's condition after obtaining the urine output rate (e.g., block 202 in FIG. 2). In some embodiments, the received data can include one or more of: a urine output rate, a total urine volume, a peak urine rate, a slope of a urine output rate curve, a time to peak urine output, a downward slope of urine output rate curve, a continuous diuretic infusion rate, a dome diuretic dosage, a prior response to fluid therapy, an estimated excess fluid volume, GFR, Creatinine levels, and/or any of the other data described herein.

At block 803, the method 800 can continue by determining whether a fluid therapy system, such as the fluid therapy system 100 of FIG. 1, recommends ending the patient's fluid therapy, as described in further detail with reference to FIGS. 1 and 2. If the system does not recommend ending the patient's fluid therapy (blocks 803, “NO”), the method 800 can include returning to block 802 and/or repeating one or more of the blocks 802, 803. If the system recommends ending the patient's fluid therapy (block 803, “YES”), the method 800 can continue to block 804.

At block 804, the method 800 can continue by using one or more models to determine information associated with continuing the patient's therapy. For example, one or more of the models can be configured to characterize the system's recommendation to end the therapy, such as by providing a confidence interval associated with the recommendation to end the patient's fluid therapy. The characterization/confidence interval can be based at least partially on the data received in block 802. For example, the model(s) can be configured/trained to compare the data received in block 802 with historical/training data from one or more other patients, and identify instance(s) where others patients having generally similar or identical treatment profiles continued receiving fluid therapy after a recommendation to end the fluid therapy. Additionally, or alternatively, one or more of the models can be configured to predict an amount of fluid that may be removed from the patient if the patient's fluid therapy continues. In these and other embodiments, one or more of the models can be configured to predict a remaining duration of the therapy that would be performed if the therapy continues.

At block 805, the method 800 can continue by determining whether to end the patient's therapy. In some embodiments, the determination can be made by one or more of the models (block 804) and based at least partially on at least some of the predicted information associated with continuing the therapy (block 804). In other embodiments, the determination can be made by a user, for example, after evaluating at least some of the predicted information associated with continuing the therapy (block 804). If the determination is to continue the patient's therapy (block 805, “NO”), the method 800 can continue to block 806. If the determination is to end the patient's therapy (block 805, “YES”), the method can continue to block 808.

At block 806, the method 800 can continue by receiving an input associated with the determination to continue the therapy. For example, the input can include an input provided by the user instructing the system 100 to continue the patient's therapy. Additionally, or alternatively, the input can include urine output data and/or other data associated with the continued patient therapy. In some embodiments, after receiving the input (block 806), the method 800 can return to block 802 and/or repeat one or more of the blocks 802-805.

At block 808, the method 800 can continue by receiving an input associated with the end of the therapy. For example, the input can include an input provided by the user instructing the system 100 to end the patient's therapy, or the therapy can be ended automatically. Additionally, or alternatively, the input can include urine output data and/or other data associated with the end patient therapy.

The method 810 of FIG. 8B is generally directed to determining a patient's readmission risk in accordance with embodiments of the present technology. The method 810 can begin at block 812 by receiving data associated with a patient. Block 812 can be generally similar or identical to block 802 of FIG. 8A. The received data can be associated with the patient's baseline condition, and/or the patient's condition after obtaining the urine output rate (e.g., block 202 in FIG. 2). In some embodiments, the received data can include one or more of: a urine output rate, a total urine volume, a peak urine rate, a slope of a urine output rate curve, a time to peak urine output, a downward slope of urine output curve, a continuous diuretic infusion rate, a home diuretic dosage, a prior response to fluid therapy, an estimated excess fluid volume, GFR, Creatinine levels, and/or any of the other data described herein.

At block 814, the method 810 can continue by determining whether a patient's fluid therapy has ended. If the patient's fluid therapy is continuing (block 814, “NO”), the method 800 can return to block 812 and repeat one or more of the blocks 812, 814. If the patient's fluid therapy has ended (block 814, “YES”), the method can continue to block 816.

At block 816, the method 810 can continue by receiving information associated with a treatment profile of the patient. The patient's treatment profile can include, for example, total amounts of diuretic and/or hydration fluid administered, total urine output, one or more lab test results, other patient parameters (e.g., heart rate, respiratory rate, blood pressure, and the like) at the end of therapy, and/or any other data described herein. In at least some embodiments, the patient's treatment profile can include aggregated and/or time-series values for one or more of the data/data types received in block 812.

At block 818, the method 810 can continue by using one or more models to predict a timeframe for and/or a likelihood of patient readmission. The timeframe and/or the likelihood of patient readmission can be based at least partially on the data received in block 812 and/or block 816. For example, the model(s) can be configured/trained to compare the data received in block 812 and/or block 816 with historical/training data and/or treatment profiles from one or more other patients and identify timeframes(s) and/or readmission rate(s) for patients having generally similar or identical treatment profiles. Block 818 can further include providing the predicted timeframe and/or likelihood of patient readmission to a user (e.g., a clinician, practitioner, and the like)

The method 820 of FIG. 8C is generally directed to determining information associated with outpatient oral diuretic dosing following treatment. The method 820 can begin at block 822 by receiving data associated with a patient. Block 822 can be generally similar or identical to block 802 of FIG. 8A. The received data can be associated with the patient's baseline condition, and/or the patient's condition after obtaining the urine output rate (e.g., block 202 in FIG. 2). In some embodiments, the received data can include one or more of: a urine output rate, a total urine volume, a peak urine rate, a slope of a urine output rate curve, a time to peak urine output, a downward slope of urine output curve, a continuous diuretic infusion rate, a dome diuretic dosage, a prior response to fluid therapy, an estimated excess fluid volume, GFR, Creatinine levels, and/or any of the other data described herein.

At block 824, the method 820 can continue by determining whether a patient's fluid therapy has ended. Block 824 can be generally similar or identical to block 814 of FIG. 8B. If the patient's fluid therapy is continuing (block 824, “NO”), the method 800 can return to block 822 and repeat one or more of the blocks 822, 824. If the patient's fluid therapy has ended (block 824, “YES”), the method can continue to block 826.

At block 826, the method 820 can continue by receiving information associated with a treatment profile of the patient. Block 826 can be generally similar to or the same as block 816 of FIG. 8B. For example, the patient's treatment profile can include, for example, total amounts of and/or hydration fluid administered, total urine output, lab test results, other patient parameters (e.g., heart rate, respiratory rate, blood pressure, and the like) at the end of therapy, and/or any other data described herein. In at least some embodiments, the patient's treatment profile can include aggregated and/or time-series values for one or more of the data/data types received in block 822.

At block 828, the method 820 can continue by using one or more models to match the patient to one or more other patients having generally similar or identical treatment profiles. For example, one or more of the models can be configured to compare the information associated with the patient's treatment profile (block 826) with other information associated with one or more of other treatment profiles (e.g., of former patients) and determine the similarity between these information.

At block 829, the method 820 can continue by using one or more of the models to determine an oral diuretic dosage. Determining the oral diuretic dosage can be determined based at least partially on oral diuretic dosing information associated with the generally similar or identical treatment profiles identified in block 828. In some embodiments, the generally similar or identical treatment profiles can include rehospitalization/readmission information, and block 829 can include using the model(s) to determine the oral diuretic dosage based at least partially on the rehospitalization/readmission information.

FIG. 8D is a flow diagram of a method 830 for informing treatment escalation for patients that “fail fast” or otherwise do not respond to fluid therapy, in accordance with embodiments of the present technology. In some embodiments, the method 830 can identify patients that may not benefit from fluid therapy and/or that may require additional treatment beyond fluid therapy. The method 830 can begin at block 832 by receiving data associated with the patient. Block 832 can be generally similar or identical to block 802 of FIG. 8A. The received data can be associated with the patient's baseline condition, and/or the patient's condition after obtaining the urine output rate (e.g., block 202 in FIG. 2). In some embodiments, the received data can include one or more of: a urine output rate, a total urine volume, a peak urine rate, a slope of a urine output rate curve, a time to peak urine output, a downward slope of urine output curve, a continuous diuretic infusion rate, a dome diuretic dosage, a prior response to fluid therapy, an estimated excess fluid volume, GFR, creatinine levels, and/or any of the other data described herein.

At block 833, the method 830 can continue by using one or more models to determine a likelihood that the patient's therapy will be stopped. The likelihood that the patient's therapy will be stopped can be based at least partially on the data received in block 832. For example, the model(s) can be configured/trained to compare the data received in block 832 with historical/training data from one or more other patients, and identify instance(s) where the fluid therapy for other patients having generally similar or identical treatment profiles was stopped. In some embodiments, block 833 can include determining the likelihood that the patient's therapy will be stopped within a time period, such as within at least 10 minutes, 1 hour, 6 hours, or within another suitable time period.

At block 834, the method 830 can continue by determining whether the likelihood (block 833) exceeds a first stop therapy threshold. The first stop therapy threshold can include a first probability greater than 50%, 60%, 70%, etc. If the likelihood is less than or equal to the first stop therapy threshold (block 834, “NO”), the method 830 can return to block 832 and/or repeat one or more of the blocks 832-834. If the likelihood is greater than the first stop therapy threshold (block 834, “YES”), the method 830 can continue to block 835.

At block 835, the method 830 can continue by determining whether the likelihood (Block 833) exceeds a second stop therapy threshold. The second stop therapy threshold can be greater than the first stop therapy threshold. For example, the second stop therapy threshold can include a second probability greater than the first probability, such as at least 75%, 85%, 95%, etc. If the likelihood is greater than the second stop therapy threshold, the method 330 can continue to block 836 and the patient's therapy can be automatically stopped, or a message can be sent to the user suggesting that therapy be stopped manually. Whether or not the likelihood is greater than the second stop therapy threshold, the method 830 can continue to block 837.

At block 837, the method 830 can continue by receiving information associated with the patient's treatment profile. Block 837 can be generally similar or identical to block 816 of FIG. 8B and/or block 826 of FIG. 8C. For example, the patient's treatment profile can include, for example, total amounts of and/or hydration fluid administered, total urine output, lab test results, other patient parameters (e.g., heart rate, respiratory rate, blood pressure, and the like) at the end of therapy, and/or any other data described herein. In at least some embodiments, the patient's treatment profile can include aggregated and/or time-series values for one or more of the data/data types received in block 832.

At block 838, the method 830 can continue by using one or more of the models to match the patient to one or more other patients having at least generally similar or identical treatment profiles. Block 838 can be at least generally similar or identical to block 828 of FIG. 8C. For example, one or more of the models can be configured to compare the information associated with the patient's treatment profile (block 837) with other information associated with one or more of other treatment profiles (e.g., of former patients) and determine the similarity between these information.

At block 839, the method 830 can continue by providing a user with information associated with one or more treatment outcomes of one or more of the other patients (block 838). The information can include, for example, subsequent treatment received after the end of fluid therapy, risk of rehospitalization/readmission, urine output data at the end of therapy, and/or other suitable information. In some embodiments, the provided information can assist the user in determining whether to stop the patient's fluid therapy.

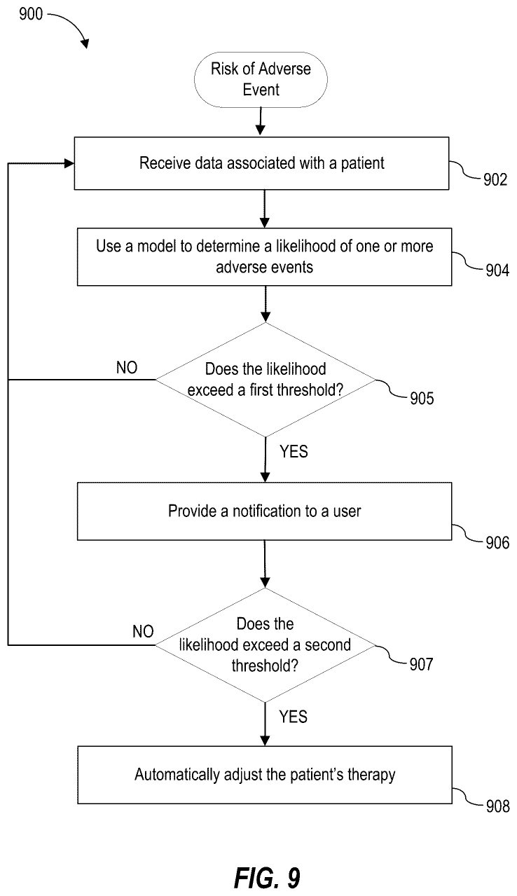

FIG. 9 illustrates a method 900 associated with predicting and/or alerting users to one or more adverse events, in accordance with embodiments of the present technology. The adverse events can include hypotension, pulmonary edema, acute kidney injury, electrolyte imbalance, heart rate abnormalities, urinary tract infection, and other adverse events. In some embodiments, the method 900 can be used to predict the risk of a patient experiencing an adverse event before therapy begins and/or as therapy progresses, alert a user if a certain risk threshold is met, and/or stop therapy automatically if the highest risk threshold is met. The method 900 can include one or more models trained using clinical/historical patient data, as described with reference to FIG. 4A. For example, one or more of the models can be trained based on patient data include one or more of the following: systolic and/or diastolic blood pressure, GFR, O₂ saturation, heart rate, lung congestion, urine output rate and volume, therapy duration, continuous diuretic infusion rate, home diuretic dosage, electrolyte profile, occurrence and/or type of adverse event(s), and/or other suitable data described herein. Generally, based on the data associated with a patient, one or more of the models can be configured to adjust the diuretic dosage rate, adjust the hydration rate, slow and/or stop therapy, and/or warn the user of the patient risk.

The method 900 can begin at block 902 by receiving data associated with the patient. Block 902 can be generally similar or identical to block 412 of FIG. 4B. The received data can be associated with the patient's baseline condition, and/or the patient's condition after obtaining the urine output rate (e.g., block 202 in FIG. 2). In some embodiments, the received data can include one or more of: systolic blood pressure, diastolic blood pressure, creatinine levels, O₂ sat, lung congestion (e.g., chest x-ray, ReDS), urine output rate, and/or any of the other data described herein.

At block 904, the method 900 can include using one or more models to determine a likelihood of one or more of adverse events. The adverse events can include any of the adverse events described herein. The likelihood of individual ones of the adverse events can be predicted based at least partially on the data received in block 902. For example, the model(s) can be configured/trained to compare the data received in block 902 with historical/training data from one or more other patients, and identify instance(s)/rate(s) at which others patients having generally similar or identical treatment profiles experienced one or more of the adverse events. In some embodiments, determining the59econdhood of one or more of the adverse events includes predicted a time until one or more of the adverse events and/or a time range in which one or more of the adverse events is expected to occur.

At block 905, the method 900 can continue by comparing the likelihood of one or more of the adverse events to a first adverse event threshold. The first adverse event threshold can include a first probability of up to 30%, 40%, 50%, 60%, etc. In some embodiments, the first adverse event threshold can include probabilities specific to individual ones of the adverse events, such that block 905 can include comparing the likelihood for a given adverse event to a probability associated with the given adverse event. If the likelihood of one or more of the adverse events is less than or equal to the first adverse event threshold (block 905, “NO”), the method 900 can return to block 902 and/or repeat one or more of the blocks 902-905. If the likelihood of or more of the adverse events is greater than the first adverse event threshold (block 905, “YES”), the method 900 can continue to block 906.

In block 906, the method 900 can continue by providing a notification to a user. The notification can include an alert, a sound, a message, or another suitable notification. In at least some embodiments, the notification can identify the adverse event and the associated likelihood that exceeded the first adverse event threshold (block 905), and/or recommend one or more interventions expected to reduce the likelihood or severity of the predicted adverse event.

In block 907, the method 900 can continue by comparing the likelihood of one or more of the adverse events (block 904) to a second adverse event threshold. The second adverse event threshold a second probability greater than the first probability. For example, the second probability can be at least 60%, 65%, 70%, 80% 90%, etc. In some embodiments, the second adverse event threshold can include probabilities specific to individual ones of the adverse events, such that block 907 can include comparing the likelihood for a given adverse event to a probability associated with the given adverse event. If the likelihood of one or more of the adverse events is less than or equal to the second adverse event threshold (block 907, “NO”), the method 900 can return to block 902 and/or repeat one or more of the blocks 902-907. If the likelihood of one or more of the adverse events is greater than the first adverse event threshold (block 907, “YES”), the method 900 can continue to block 908.

At block 908, the method 900 can continue by automatically adjusting the patient's therapy. Automatically adjusting the patient's therapy can include increasing or decreasing the diuretic dosage rate, increase or decreasing the hydration rate, stopping therapy, or another adjustment to the patient's therapy. In some embodiments, the adjustment to the patient's therapy can be responsive to the type of one or more of the adverse events. For example, if the likelihood of pulmonary edema exceeds the second adverse event threshold, block 908 can include stopping delivery of hydration fluid. As another example, if the likelihood of hypotension exceeds the second adverse event threshold, block 908 can include increasing the hydration fluid rate. As a further example, if the likelihood of electrolyte imbalance exceeds the second adverse event threshold, block 908 can include stop delivery of the diuretic. Alternately, the method 900 can continue by displaying a message to prompt the user to adjust the patient's therapy.

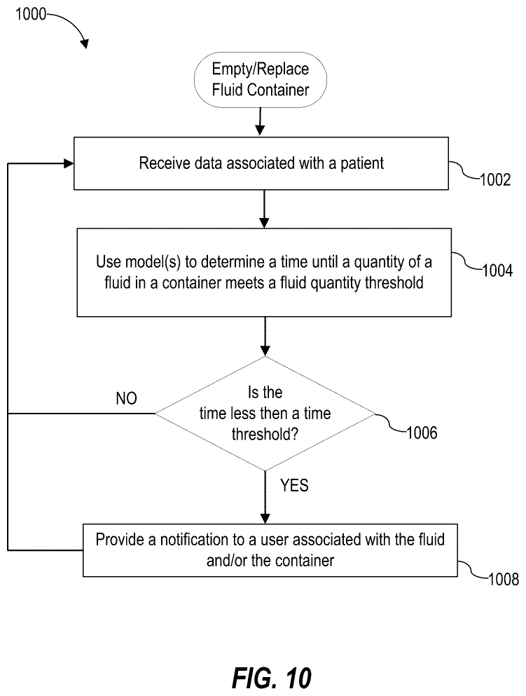

FIG. 10 illustrates a method 1000 associated with fluid levels of a fluid therapy system in accordance with embodiments of the present technology. In some embodiments, the method 1000 can be used to predict a time until a urine bag is full, a hydration fluid source is empty, and/or a diuretic source is empty, for example, so that these sources/bags can be emptied/filled to reduce or prevent interruptions to the patient's fluid therapy. The method 1000 can include one or more models trained using clinical/historical patient data, as described with reference to FIG. 4A. For example, one or more of the models can be trained based on patient data include one or more of the following: urine output rate, total urine volume, peak urine rate, slope of urine output rate curve, time to peak urine output, downward slope of urine output curve, diuretic ramp, continuous infusion dose, saline infusion rate, and/or other suitable data described herein. Generally, based on the data associated with a patient, one or more of the models can be configured to adjust the diuretic dosage rate, adjust the hydration rate, slow and/or stop therapy, and/or warn the user of the patient risk.

The method 1000 can begin at block 1002 by receiving data associated with a patient. Block 1002 can be generally similar or identical to block 412 of FIG. 4B. The received data can be associated with the patient's baseline condition, and/or the patient's condition after obtaining the urine output rate (e.g., block 202 in FIG. 2). In some embodiments, the received data can include one or more of: urine output rate, total urine volume, peak urine rate, slope of urine output rate curve, time to peak urine output, and/or any of the other data described herein.

At block 1004, the method 1000 can continue by using one or more models to determine a time until a quantity of a fluid in a container meets a fluid quantity threshold. The time can be determined based at least partially on the data received in block 1002. For example, the model(s) can be configured/trained to compare the data received in block 1002 with historical/training data from one or more other patients, and identify time(s) until a quantity of fluid in a container used to treat other patients having generally similar or identical treatment profiles met an associated fluid quantity threshold. The fluid quantity threshold can be up to 5%, 10%, 20%, 25%, 50%, 75%, 80%, 90%, or 100% of a volume of the container. In some embodiments, the fluid can include urine, the container can include one or more collection containers configured to hold urine, such as the container 112 of FIG. 1, and the fluid quantity threshold can be associated with at least one of the collection containers being full or otherwise at maximum capacity (e.g., contains 100% capacity). Additionally, or alternatively, the fluid can include hydration fluid, the container can include one or more hydration fluid sources, such as the fluid source 122 of FIG. 1, and the fluid quantity threshold can be associated with at least one of the hydration fluid sources being empty (e.g., 100% of the capacity has been removed). In these and other embodiments, the fluid can include a diuretic, the container can include one or more diuretic sources, such as the diuretic source 134 of FIG. 1, and the fluid quantity threshold can be associated with at least one of the diuretic sources being empty.

At block 1006, the method 1000 can continue by determining whether the time (block 1004) is less than a time threshold. The time threshold can be at least 1 minute, 5 minutes, 30 minutes, 1 hour, any time therebetween, or another suitable time. If the time is greater than or equal to the time threshold (block 1006, “NO”), the method can return to block 1002 and/or repeat one or more of the blocks 1002, 1004. If the time is less than the time threshold (block 1006, “YES”), the method 1000 can continue to block 1008.

At block 1008, the method 1000 can continue by providing a notification to a user associated with the fluid and/or the container (block 1004). In some embodiments, the fluid includes urine and/or the container include one or more of the collection containers, and the notification can include prompting the user to empty at least one of the collection containers, such as any of collection containers predicted to be full within the time (block 1004). Additionally, or alternatively, the fluid includes hydration fluid and/or the container includes a fluid source, and the notification includes prompting the user to replace at least one of the fluid sources, such as any of the source bags determined to be empty within the time (block 1004). In these and other embodiments, the fluid includes diuretic and/or the container includes a diuretic source, and the notification includes prompting the user to replace at least one of the diuretic sources, such as any of the diuretic sources determined to be empty within the time (block 1004). In some embodiments, after providing the notification and/or after the user empties/replaces at least one container, the method 1000 can return to block 1002 and/or repeat one or more of the blocks 1002-1008.

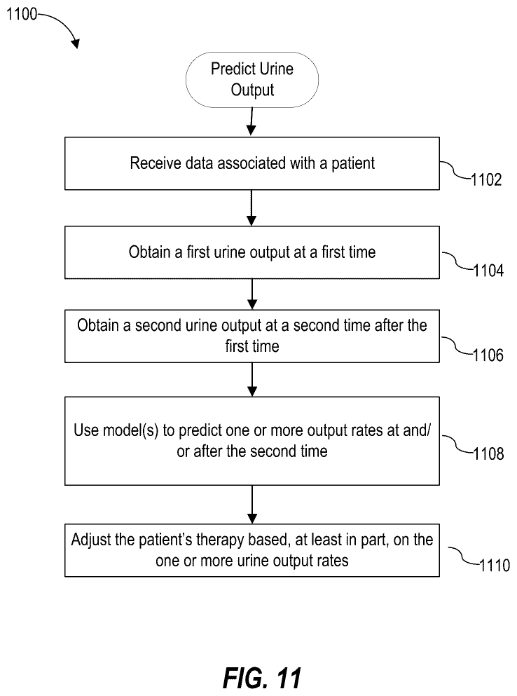

FIG. 11 illustrates a method 1100 associated with predicting a patient's urine output in accordance with embodiments of the present technology. In many instances, patient urine output data is continuous, e.g., because the patient has a Foley catheter and urine in the bladder is removed from the patient without voluntary action (e.g., voluntary urination). However, in some instances patient urine output data is discontinuous and/or intermittent, e.g., because the patient has a condom catheter or other non-Foley style catheter such that the urine output data is obtained only after the patient urinates. For patients with discontinuous urine output data, it can be difficult to adjust their fluid therapy because the available urine output data is based on boluses of urine obtained from the patient intermittently. However, the method 1100 can be used to interpolate or otherwise predict a patient's urine output before, during, and/or after one or more intermittent boluses of urine, for example, so that the patient's fluid therapy can be adjusted based, at least in part, on one or more of the boluses or urine and/or the predicted urine output between boluses. The method 1100 can include one or more models trained using clinical/historical patient data, as described with reference to FIG. 4A. For example, one or more of the models can be trained based on patient data include one or more of the following: one or more urine analyte values, urine sodium concentration, urine output rate, total urine volume, peak urine rate, slope of urine output rate curve, time to peak urine output, downward slope of urine output curve, diuretic ramp, continuous infusion dose, saline infusion rate, and/or other suitable data described herein. Generally, based on the data associated with a patient, one or more of the models can be configured to predict a urine output between individual boluses of urine and, in some embodiments, may adjust the diuretic dosage rate, adjust the hydration rate, slow and/or stop therapy, and/or warn the user of the patient risk based, at least in part, on the predicted urine output.

The method 1100 can begin at block 1102 by receiving data associated with a patient. Block 1102 can be generally similar or identical to block 412 of FIG. 4B. The received data can be associated with the patient's baseline condition, and/or the patient's condition after obtaining the urine output rate (e.g., block 202 in FIG. 2). In some embodiments, the received data can include one or more of: one or more urine analyte values, urine sodium concentration, urine output rate, total urine volume, peak urine rate, slope of urine output rate curve, time to peak urine output, and/or any of the other data described herein.

At block 1104, the method 1000 can continue by obtaining a first urine output of the patient at a first time. As noted above, the first urine output can be a first discontinuous and/or intermittent urine output, e.g., based at least in part on one or more boluses of urine excreted from (e.g., voluntarily urinated from) the patient. In some embodiments, obtaining the first urine output can include obtaining a volume and/or a weight of urine, and/or an amount and/or concentration of sodium in the urine.

At block 1106, the method 1000 can continue by obtaining a second urine output of the patient at a second time after the first time. Obtaining the second urine output can be at least generally similar or identical to obtaining the first urine output (block 1104), but can occur some amount of time after obtaining the first urine output. As set forth above, the amount of time after obtaining the first urine output can be difficult to predict and, in various embodiments, can be on the order of minutes (e.g., up to 1, 2, 3, 4, 5, 10, 20, 30, etc. minutes) or hours (e.g., up to 1, 2, 3, 4, 5, 6, 7, 8, 9, 10, 11, 12, etc. hours)

At block 1108, the method 1100 can continue by using one or more models to predict one or more urine output rates at and/or after the second time. Individual ones of the urine output rates can at least approximate continuous urine output data for the patient and include an expected or predict urine output and/or urine output rate removed from the patient at and/or after the second time. Additionally, or alternatively, individual ones of the urine output rates can include a predicted or expected amount and/or concentration of sodium removed (e.g., via urine) from the patient at and/or after the second time. The urine output rates can be determined based at least partially on the data received in one or more of blocks 1102-1106. For example, the model(s) can be configured/trained to compare the data received in one or more of blocks 1102-1106 with historical/training data from one or more other patients, and identify one or more urine outputs and/or urine output rates for other patients having generally similar or identical treatment profiles. The models can predict the one or more urine output rates based on the first urine output (block 1104) and/or the second urine output (block 1106).

At block 1110, the method 1100 can continue by adjusting the patient's therapy based, at least in part, on the one or more urine output rates. The adjustments to the patient's therapy can include increasing or decreasing an amount and/or rate of diuretic and/or hydration fluid administered to the patient. In some embodiments, adjusting the patient's therapy includes recommending or suggesting (e.g., to a clinician and/or other user) one or more of the adjustments to the patient's therapy. In other embodiments, adjusting the patient's therapy includes automatically adjusting the patient's therapy.

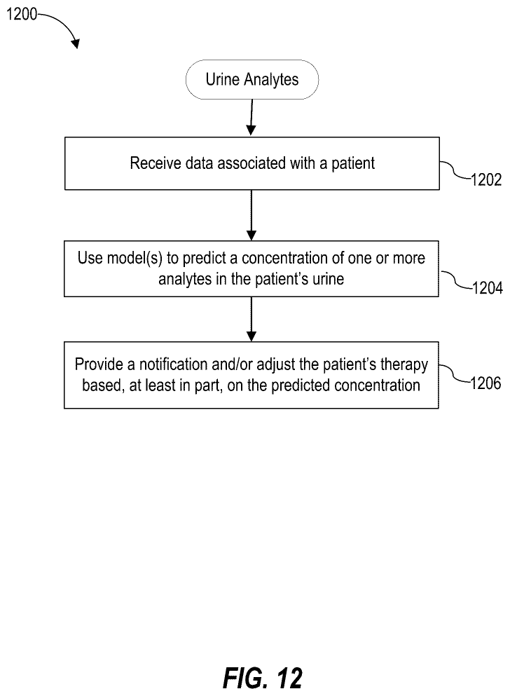

FIG. 12 illustrates a method 1200 associated with predicting an amount and/or concentration of one or more analytes in a patient's urine, in accordance with embodiments of the present technology. The method 1200 can include one or more models trained using clinical/historical patient data, as described with reference to FIG. 4A. For example, one or more of the models can be trained based on patient data to include one or more of the following: one or more lab analyte values, urine sodium concentration, urine conductivity, urine output rate, total urine volume, peak urine rate, slope of urine output rate curve, time to peak urine output, downward slope of urine output curve, diuretic ramp, continuous infusion dose, saline infusion rate, and/or other suitable data described herein. Generally, based on the data associated with a patient, one or more of the models can be configured to predict an amount and/or a concentration of sodium, potassium, and/or one or more other analytes in urine removed from the patient and, in some embodiments, may recommend adjusting the diuretic dosage rate, adjusting the hydration rate, slowing and/or stopping therapy, and/or may notify the user based, at least in part, on the predicted urine analytes.

The method 1200 can begin at block 1202 by receiving data associated with a patient. Block 1202 can be generally similar or identical to block 412 of FIG. 4B. The received data can be associated with the patient's baseline condition, and/or the patient's condition after obtaining the urine output rate (e.g., block 202 in FIG. 2). In some embodiments, the received data can include one or more of: one or more urine analyte values, urine sodium concentration, urine conductivity, urine output rate, total urine volume, peak urine rate, slope of urine output rate curve, time to peak urine output, and/or any of the other data described herein.

At block 1204, the method 1200 can continue by using one or more models to predict a concentration of one or more analytes in urine removed from the patient. The analytes can include sodium, potassium, creatine, and/or any other analytes described herein and/or any other suitable analytes and/or other compounds in urine. The predicted analyte concentrations can be determined based at least partially on the data received in block 1202, such as the patient's urine conductivity and/or urine output rate. For example, the model(s) can be configured/trained to compare the data received in block 1202 with historical/training data from one or more other patients, and identify a concentration of one or more urine analytes for other patients having generally similar or identical treatment profiles. In some embodiments, the models can be trained/configured to identify or predict one or more hydration fluid infusion rates that are expected to increase or optimize a net loss rate of one or more of the analytes (e.g., sodium).

At block 1206, the method 1200 can continue by providing a notification and/or adjusting the patient's therapy based, at least in part, on the predicted concentration (block 1204). In some embodiments, providing the notification can include notifying a user that a level of one or more of the patient's analytes are low, e.g., at or below an analyte concentration threshold. Additionally, or alternatively, providing the notification can include providing (e.g., to a user) an estimate of the volume of the patient's oral fluid intake and/or an alert that the rate of oral intake may be too high. In these and/or other embodiments, providing the notification can include notifying a user of the one or more hydration fluid infusion rates that are expected to increase or optimize a net loss rate of one or more of the analytes (e.g., sodium) In some embodiments, adjusting the therapy can include increasing or decreasing an amount and/or rate of diuretic and/or hydration fluid administered to the patient, and/or administering electrolytes, e.g., to replace or increase the concentration of one or more of the analytes, including to optimize levels of one or more analytes in the patient's fluids and/or in fluid removed from the patient. For example, if the models indicate that the patient's urine potassium is likely to be high, the patient is likely to have low serum potassium, putting the patient at risk for an adverse event; this can be prevented if therapy is adjusted to replace potassium quickly. Additionally, or alternatively, adjusting the therapy can include administering hydration fluid at the one or more hydration fluid infusion rates that are expected to increase or optimize the net loss rate of one or more of the analytes. In some embodiments, adjusting the patient's therapy includes recommending or suggesting (e.g., to a clinician and/or other user) one or more of the adjustments to the patient's therapy. In other embodiments, adjusting the patient's therapy includes automatically adjusting the patient's therapy.

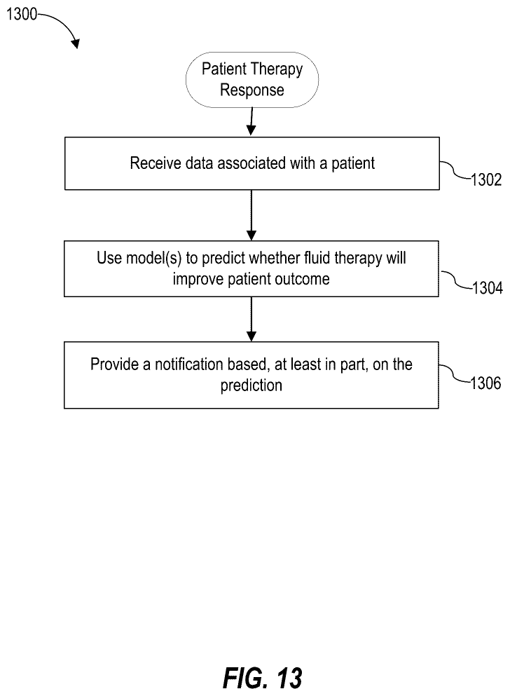

FIG. 13 illustrates a method 1300 associated with predicting an outcome of fluid therapy for a patient, in accordance with embodiments of the present technology. The method 1300 can include one or more models trained using clinical/historical patient data, as described with reference to FIG. 4A. For example, one or more of the models can be trained based on patient data include one or more of the following: one or more urine analyte values, urine sodium concentration, urine conductivity, urine output rate, total urine volume, peak urine rate, slope of urine output rate curve, time to peak urine output, downward slope of urine output curve, diuretic ramp, continuous infusion dose, saline infusion rate, and/or other suitable data described herein. Generally, based on the data associated with a patient, one or more of the models can be configured to predict whether a patient we be a responder to fluid therapy, e.g., whether fluid therapy is expected to remove a predetermined about of fluid from the patient, a predicted percent of a target fluid and/or sodium loss goal that the patient is likely to achieve via fluid therapy, and/or whether fluid therapy is otherwise expected to improve the patient's outcome.

The method 1300 can begin at block 1302 by receiving data associated with a patient. Block 1302 can be generally similar or identical to block 412 of FIG. 4B. The received data can be associated with the patient's baseline condition, and/or the patient's condition after obtaining the urine output rate (e.g., block 202 in FIG. 2). In some embodiments, the received data can include one or more of: one or more urine analyte values, urine sodium concentration, urine conductivity, urine output rate, total urine volume, peak urine rate, slope of urine output rate curve, time to peak urine output, and/or any of the other data described herein.

At block 1304, the method 1300 can continue by using one or more models to predict whether fluid therapy will improve the patient's outcome. For example, the model(s) can be configured/trained to compare the data received in block 1302 with historical/training data from one or more other patients, and identify a percentage of patients who had successful treatment outcomes from other patients having generally similar or identical treatment profiles. In at least some embodiments, block 1302 can include receiving a target fluid loss/loss rate for the patient and, in block 1304 the models can be trained/configured to predict the likelihood that fluid therapy (e.g., for a given duration, at one or more hydration fluid infusion rates, at one or more diuretic dosage rates, using one or more diuretics, etc.) will cause the patient to achieve the target fluid loss/loss rate. Additionally, or alternatively, block 1302 can include receiving a target sodium loss/loss rate and, in block 1304, the models can be trained/configured to predict the likelihood that fluid therapy will cause the patient to achieve the target sodium loss/loss rate, and/or a percent of a target fluid and/or sodium loss goal that is likely to be achieved via fluid therapy.

At block 1306, the method 1300 can continue by providing a notification based, at least in part, the prediction (block 1304). In some embodiments, providing the notification can include notifying a user about information including and/or associated with the prediction. For example, providing the notification can include notifying the user about whether fluid therapy is or is not expected to improve the patient's outcome. In at least some embodiments, for example, if the received data (block 1302) includes a target fluid loss and/or target sodium loss rate and the prediction (block 1304) indicates that fluid therapy is not expected to achieve the target fluid loss and/or the target sodium loss rate, then providing the notification can include notifying the user of this information.

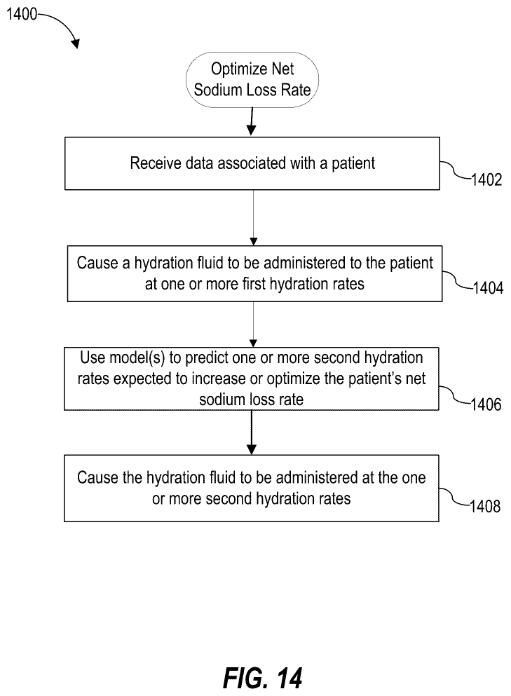

FIG. 14 illustrates a method 1400 associated with optimizing a net sodium loss rate for a patient, in accordance with embodiments of the present technology. The method 1400 can include one or more models trained using clinical/historical patient data, as described with reference to FIG. 4A. For example, one or more of the models can be trained based on patient data include one or more of the following: one or more urine analyte values, urine sodium concentration, urine conductivity, urine output rate, total urine volume, peak urine rate, slope of urine output rate curve, time to peak urine output, downward slope of urine output curve, diuretic ramp, continuous infusion dose, saline infusion rate, and/or other suitable data described herein. Generally, based on the data associated with a patient, one or more of the models can be configured to predict one or more hydration fluid administration rates that are expected to improve, or even optimize, the patient's net sodium loss rate.

The method 1400 can begin at block 1402 by receiving data associated with a patient. Block 1402 can be generally similar or identical to block 412 of FIG. 4B. The received data can be associated with the patient's baseline condition, and/or the patient's condition after obtaining the urine output rate (e.g., block 202 in FIG. 2). In some embodiments, the received data can include one or more of: one or more urine analyte values, urine sodium concentration, urine conductivity, urine output rate, total urine volume, peak urine rate, slope of urine output rate curve, time to peak urine output, and/or any of the other data described herein.

At block 1404, the method 1400 can continue by causing a hydration fluid to be administered to the patient at one or more first hydration rates. In some embodiments, the one or more first hydration rates can be based, at least in part, on the patient's urine output (e.g., urine output rate), e.g., to match a set or predetermined percentage of the patient's urine output.

At block 1406, the method 1400 can continue by using one or more models to predict one or more second hydration rates. For example, the model(s) can be configured/trained to compare the data received in block 1402 and/or block 1404 with historical/training data from one or more other patients, and identify one or more hydration rates administered to other patients having generally similar or identical treatment profiles. In at least some embodiments, the one or more second hydration rates are expected to improve or optimize the patient's net sodium loss rate.

At block 1408, the method 1400 can continue by causing the hydration fluid to be administered to the patient at the one or more second hydration rates different than the first hydration rates. In some embodiments, causing the hydration fluid to be administered includes automatically causing the hydration fluid to be administered. In other embodiments, causing the hydration fluid to be administered includes providing a user with a notification or other communication that includes the one or more second hydration rates, prompting the user to adjust the hydration fluid administration rate from the one or more first hydration rates to the one or more second hydration rates, and/or receiving an input from a user that adjusting the hydration fluid infusion from the one or more first hydration rates to the one or more second hydration rates.

### IV. Examples

Additional aspects of various embodiments of the present technology are described with reference to the following examples:

1. A method of treating a patient, the method comprising:
obtaining a urine output from the patient;
causing a diuretic to be provided to the patient at a diuretic dosage rate;
causing a hydration fluid to be provided to the patient at a hydration fluid rate; and
adjusting at least one of the diuretic dosage rate or the hydration fluid rate to thereby cause a net fluid loss from the patient,
wherein at least one of the diuretic dosage rate, the hydration fluid rate, the adjusted diuretic dosage rate, or the adjusted hydration fluid rate is determined by a model using the urine output.

2. A method of treating a patient, the method comprising:
training a model using first data received from one or more historical patients, wherein training the model includes—
   training the model to determine a diuretic dosage rate for a diuretic for delivery to the patient,
   training the model to determine a hydration fluid rate for a hydration fluid for delivery to the patient,
   training the model to determine a time at which to stop treating the patient; or
   training the model to predict a likelihood of the patient experiencing an adverse event; receiving second data from the patient; and
based at least partially on the second data, using the model to—
   determine the diuretic dosage rate,
   determine a hydration fluid rate,
   determine a time at which to stop treating the patient, or
   predict a likelihood of the patient experiencing the adverse event.

3. The method of example 2 wherein the second data includes bioimpedance spectrometry data, body temperature data, a continuous diuretic infusion rate, creatinine levels, a diastolic blood pressure, a downward slope of urine output curve, an estimated excess fluid volume, a GFR, a heart rate, a heart rate variability, a hematocrit and/or hemoglobin level, a hemodynamic measurement, a home diuretic dosage, an injected marker of hemoconcentration and/or GFR, an IVC diameter, lung congestion data, an O₂ saturation, a real-time weight of the patient, a patient therapy start weight, a peak urine rate, data associated with a prior need for therapy escalation, data associated with a prior response to fluid therapy, a respiration rate, a serum analyte concentration, a serum sodium level, data associated with signs and/or symptoms of fluid overload condition, a slope of urine output rate curve, a systolic blood pressure, a time to peak urine output, a total urine volume, one or more urine analyte levels, urine conductivity, a urine diuretic concentration, a urine output rate, and/or a urine sodium level.

4. A method of treating a patient, the method comprising:
programming a fluid therapy system to:
   obtain a urine output from the patient;
   cause a diuretic to be provided to the patient at a diuretic dosage rate;
   cause a hydration fluid to be provided to the patient a hydration fluid rate; and
   adjust at least one of the diuretic dosage rate or the hydration fluid rate to thereby cause a net fluid loss from the patient;
wherein programming the systems includes programming a model to determine at least one of the diuretic dosage rate, the hydration fluid rate, the adjusted diuretic dosage rate, or the adjusted hydration fluid rate.

5. The method of example 4, further comprising using the fluid therapy system to treat a fluid overload condition of the patient.

6. A method for optimizing a dosage rate of a diuretic provided to a patient during fluid therapy, the method comprising:
receiving data associated with a patient;
receiving an expected diuretic dosage rate for the patient from a model trained to compare the received data with historical data and/or training data associated with one or more other patients that have a generally similar treatment profile as the patient, wherein the model is configured to determine the expected diuretic dosage rate based, at least in part, on the received data and one or more historical diuretic dosage rates administered to the one or more other patients;
causing a diuretic to be administered to the patient at a dosage rate at or near the expected diuretic dosage rate; and
based, at least in part, on a response of the patient to the administration of the diuretic, adjusting the dosage rate of the diuretic.

7. The method of clause 6, wherein adjusting the dosage rate of the diuretic includes increasing or decreasing the dosage rate of the diuretic

8. The method of clause 6, further comprising, prior to adjusting the dosage rate of the diuretic, providing a notification to a user including a suggested increase or decrease to the dosage rate of the diuretic.

9. The method of any of clauses 6-8, wherein adjusting the diuretic dosage rate includes adjusting the diuretic dosage rate based, at least in part, on a difference between a measured urine output of the patient and a desired urine output of the patient.

10. The method of any of clauses 6-9, wherein causing the diuretic to be administered includes causing an optimum diuretic to be administered, and wherein the method further comprises receiving the optimum diuretic from the model and/or one or more other models trained to compare the received data with historical data and/or training data associated with one or more other patients that have an at least generally similar treatment profile as the patient and configured to determine the expected diuretic dosage rate based, at least in part, on the received data and one or more historical diuretics administered to the one or more other patients.

11. The method of any of clauses 6-10, further comprising receiving, from the model and/or one or more other models, a likelihood of success for one or more therapy escalation options, including administration of an additional loop diuretic, temporary fluid matching, and/or thiazide administration, wherein the model and/or the one or more other models are configured to compare the received data with historical data and/or training data associated with one or more other patients that have an at least generally similar treatment profile as the patient to determine the likelihood of success based, at least in part, on the received data and one or more historical instances and/or success rates for the one or more therapy escalation options administered to the one or more other patients.

12. A method for optimizing delivery of a hydration fluid to a patient during fluid therapy, the method comprising:
receiving data associated with a patient;
receiving a predicted hydration fluid infusion intolerance risk of the patient, wherein the predicted hydration fluid infusion intolerance risk is received from a model trained to compare the received data with historical data and/or training data associated with one or more other patients that have an at least generally similar treatment profile as the patient, and wherein the model is configured to determine the predicted hydration fluid infusion intolerance risk based, at least in part, on the received data and one or more instances of hydration fluid infusion intolerance associated with the one or more other patients; and
causing a hydration fluid to be administered to the patient at a hydration rate selected based, at least in part, on the predicted hydration fluid infusion intolerance risk of the patient.

13. The method of clause 12, wherein the predicted hydration fluid infusion intolerance risk includes a likelihood that a hydration fluid infusion rate matching the patient's urine output increases a risk of one or more adverse events.

14. The method of clause 12 or 13, wherein the hydration rate is less than the patient's urine output rate.

15. The method of clause 12 or 13, wherein the hydration rate is greater than the patient's urine output rate.

16. The method of any of clauses 12-15, wherein, if the predicted hydration fluid infusion intolerance risk is above a high risk threshold, no hydration fluid is administered or the hydration rate matches up to a first amount of the patient's urine output.

17. The method of clause 16, wherein the first amount is 100 mL.

18. The method of clause 16 or 17, wherein the high risk threshold is at least 70%.

19 The method of any of clauses 12-18, wherein if the predicted hydration fluid intolerance risk is at or below a low risk threshold, the hydration rate matches at least a second amount of the patient's urine output.

20 The method of clause 19 wherein the second amount is 500 mL.

21. The method of clause 18 or 19, wherein the low risk threshold is up to 30%.

22. The method of any of clauses 12-21, wherein, if the predicted hydration fluid infusion intolerance risk is between a low risk threshold and a high risk threshold, the hydration rate matches up to a third amount of the patient's urine output.

23 The method of clause 22 wherein the third amount is 350 mL.

24. The method of any of clauses 12-23, further comprising decreasing the hydration rate in response to a risk of hypotension exceeding a hypotension threshold.

25 A method for determining a readmission risk of a fluid therapy patient, the method comprising:
receiving data associated with a fluid therapy provided to the patient; and
after the fluid therapy has ended—
   receiving information associated with a treatment profile of the patient; and
   receiving a predicted timeframe for patient readmission and/or a likelihood of patient readmission,
wherein the predicted timeframe and/or the likelihood are received from a model trained to compare the received data with historical data and/or training data associated with one or more other patients that have an at least generally similar treatment profile as the patient, and
wherein the model determines the predicted timeframe for patient readmission and/or the likelihood of patient readmission based, at least in part, on the received data and readmission timeframes and/or readmission rates for the one or more other patients.

26. The method of clause 25, wherein receiving information associated with the treatment profile includes a total amount of diuretic and/or hydration fluid administered, a total urine output, one or more lab test results, a patient heart rate, a patient respiratory rate, and/or a patient blood pressure.

27. A method for outpatient diuretic dosing, the method comprising:
after a fluid therapy provided to a patient has ended—
   receiving information associated with the fluid therapy; and
   receiving an oral diuretic dosage for the patient, wherein the oral diuretic dosage is received from a model trained to compare the received information with historical data and/or training data associated with one or more other patients that have an at least generally similar treatment profile as the patient,
   wherein the model determines the oral diuretic dosage for the patient based, at least in part, on the received data and one or more oral diuretic dosages proscribed to the one or more other patients.

28. A method for predicting an adverse event risk before and/or during fluid therapy, the method comprising:
receiving data associated with a patient before and/or during the fluid therapy;
receiving a likelihood of an adverse event from a model trained to compare the received data with historical data and/or training data associated with one or more other patients so as to identify individual ones of the one or more other patients that have an at least generally similar treatment profile as the patient, wherein the model determines the likelihood of an adverse event based, at least in part, on the received data and adverse event occurrence data associated with the one or more other patients;
when the likelihood of the adverse event exceeds a first threshold, providing a notification to a user; and
when the likelihood of the adverse event exceeds a second threshold, automatically adjusting the patient's therapy.

29 The method of clause 28, wherein automatically adjusting the patient's therapy includes increasing or decreasing a dosage rate of a diuretic administered to the patient, increasing or decreasing a rate at which hydration fluid is administered to the patient, and/or stopping the patient's therapy.

30 The method of clause 28 or 29, wherein the automatic adjustment to the patient's therapy is based, at least in part, on a type of the adverse event.

31. The method of any of clauses 28-30, wherein the adverse event includes pulmonary edema, and wherein automatically adjusting the patient's therapy includes stopping delivery of hydration fluid.

32 The method of any of clauses 28-31, wherein the adverse event includes an electrolyte imbalance and wherein automatically adjusting the patient's therapy includes stopping delivery of a diuretic.

33. The method of any of clauses 28-32, wherein the second threshold is higher than the first threshold.

34 The method of any of clauses 28-33, wherein the first threshold is up to 60% and wherein the second threshold is at least 65%.

35. A method associated with fluid levels of a fluid therapy system, the method comprising:
receiving data associated with a patient;
receiving an expected time until a quantity of a fluid in a container of the fluid therapy system meets a fluid quantity threshold from a model trained to compare the received data with historical data and/or training data associated with one or more other patients that have a generally similar treatment profile as the patient, wherein the model is configured to determine the expected time based, at least in part, on the received data and one or more historical times for the one or more other patients; and
when the expected time is less than a time threshold, providing a notification to a user associated with the fluid and/or the container.

36. The method of clause 35 wherein the fluid includes a hydration fluid and the container includes a hydration fluid source.

37. The method of clause 35 or 36 wherein the fluid includes a diuretic and the container includes a diuretic source.

38. The method of any of clauses 35-37 wherein the fluid includes urine and the container includes a collection container.

39. The method of any of clauses 35-38 wherein the fluid quantity threshold is up to 5%, 10%, 20%, 25%, 50%, 75%, 80%, 90%, or 100% of a volume of the container.

40. The method of any of clauses 35-39 wherein providing the notification includes prompting the user to replace the container with another container.

41. The method of any of clauses 35-40 wherein the container includes a hydration fluid source and wherein providing the notification includes prompting the user to replace the hydration fluid source with a new hydration fluid source.

42. The method of any of clauses 35-41 wherein the container includes a diuretic source, and wherein providing the notification includes prompting the user to replace the diuretic source.

43. The method of any of clauses 35-42 wherein the container includes a urine collection container, and wherein providing the notification includes prompting the user to empty the urine collection container.

44. A method for predicting a patient's response to fluid therapy, the method comprising:
receiving data associated with a patient;
receiving a prediction of whether fluid therapy will improve the patient's outcome from a model trained to compare the received data with historical data and/or training data associated with one or more other patients that have a generally similar treatment profile as the patient, wherein the model is configured to make the prediction based, at least in part, on the received data and one or more historical patient outcomes for the one or more other patients; and
providing a notification to a user associated with the prediction.

45. The method of clause 44 wherein receiving the data includes receiving a target fluid loss and/or a target fluid loss rate for the patient, and wherein receiving the prediction includes receiving a prediction whether fluid therapy will cause the patient to achieve the target fluid loss and/or the target fluid loss rate.

46 The method of clause 44 or 45 wherein receiving the data includes receiving a target sodium loss for the patient, and wherein receiving the prediction includes receiving a prediction whether fluid therapy will cause the patient to achieve the target sodium loss for the patient.

47. The method any of clauses 44-46 wherein receiving the data includes receiving a target fluid and/or sodium loss for the patient, and wherein receiving the prediction includes receiving a percentage of the target fluid and/or sodium loss expected to be removed from the patient via fluid therapy.

48. A method for predicting a patient's urine output, the method comprising:
receiving data associated with a patient;
obtaining a urine output of the patient at a time;
receiving one or more urine output rates at and/or after the time from a model trained to compare the received data with historical data and/or training data associated with one or more other patients that have a generally similar treatment profile as the patient, wherein the model is configured to determine the one or more urine output rates based, at least in part, on the received data and one or more historical times for the one or more other patients; and
adjusting the patient's fluid therapy based, at least in part, on the one or more urine output rates.

49 The method of clause 48 wherein the urine output is discontinuous.

50. The method of clause 48 or 49 wherein obtaining the urine output includes obtaining the urine output via a non-Foley style catheter.

51. The method of any of clauses 48-50 wherein the one or more urine output rates at least approximate continuous urine output data for the patient

52. The method of any of clauses 48-51 wherein the one or more urine output rates include an expected or predict urine output and/or urine output rate removed from the patient at and/or after the time.

53. The method of any of clauses 48-52 wherein adjusting the patient's fluid therapy includes increasing or decreasing an amount and/or rate of a hydration fluid and/or a diuretic administered to the patient.

54. The method of any of clauses 48-53 wherein the urine output is a second urine output and the time is a second time, the method further comprising obtaining a first urine output at a first time before the second time, wherein the one or more urine output rates are predicted based, at least in part, on the first urine output and the second urine output.

55. A method for predicting a concentration of one or more analytes in urine removed from a patient, the method comprising:
receiving data associated with a patient;
receiving a predicted concentration of one or more analytes in urine removed from the patient from a model trained to compare the received data with historical data and/or training data associated with one or more other patients that have a generally similar treatment profile as the patient, wherein the model is configured to make the prediction based, at least in part, on the received data and one or more historical urine analyte concentrations for the one or more other patients; and
providing a notification and/or adjusting the patient's therapy based, at least in part, on the predicted concentration.

56. The method of clause 55 wherein the one or more analytes include sodium, potassium, and/or creatine.

57. The method of clause 55 or 56 wherein adjusting the patient's therapy includes increasing or decreasing an amount and/or rate of a hydration fluid and/or a diuretic administered to the patient.

58. The method of any of clauses 55-57 wherein the one or more analytes include sodium, and wherein adjusting the patient's therapy includes increasing or decreasing an amount and/or rate of a hydration fluid and/or a diuretic administered to the patient to optimize sodium removal from the patient.

59. The method of any of clauses 55-58 wherein adjusting the patient's therapy includes causing replacement analytes to be administered to the patient.

60. The method of any of clauses 55-59 wherein providing the notification includes notifying a user that a level of one or more of the patient's analytes is at or below an analyte concentration threshold.

61. The method of any of clauses 55-60 wherein adjusting the patient's therapy includes causing the administration of a hydration fluid and/or a diuretic to be stopped.

62. A method for predicting whether a patient's diuretic dosage will be re-ramped during fluid therapy, the method comprising:
receiving data associated with a patient;
receiving a predicted likelihood that the patient's diuretic dosage will be re-ramped from the patient from a model trained to compare the received data with historical data and/or training data associated with one or more other patients that have a generally similar treatment profile as the patient, wherein the model is configured to make the prediction based, at least in part, on the received data and one or more re-ramp occurrence rates for the one or more other patients; and
when the predicted likelihood exceeds a re-ramp threshold, causing the patient's diuretic dosage to be re-ramped.

63. The method of clause 62, further comprising providing a notification to a user prior to causing the patient's diuretic dosage to be re-ramped.

64. The method of clause 62 or clause 63 wherein causing the patient's diuretic dosage to be re-ramped includes increasing a dosage rate of the diuretic from a first rate to one or more second rates.

65. A method for optimizing a patient's net sodium loss rate, the method comprising:
receiving data associated with a patient;
causing a hydration fluid to be administered to the patient at one or more first hydration rates;
receiving one or more second hydration rates configured to increase or optimize a net sodium loss rate of the patient from a model trained to compare the received data with historical data and/or training data associated with one or more other patients that have a generally similar treatment profile as the patient, wherein the model is configured to identify the one or more second hydration rates based, at least in part, on the received data and net sodium loss rates for the one or more other patients; and
causing the hydration fluid to be administered at the one or more second hydration rates.

66. The method of clause 65 wherein the one or more first hydration rates are based, at least in part, on a urine output rate of the patient.

67. The method of clause 65 or 66 wherein the one or more first hydration rates are a predetermined percentage of a urine output rate of the patient.

68. The method of any of clauses 65-67 wherein causing the hydration fluid to be administered at the one or more second hydration rates includes automatically causing the hydration fluid to be administered at the one or more second hydration rates.

69 The method of any of clauses 65-68 wherein causing the hydration fluid to be administered at the one or more second hydration rates includes receiving an input from a user that causes the hydration fluid to be administered at the one or more second hydration rates.

### V. Conclusion

It will be apparent to those having skill in the art that changes may be made to the details of the above-described embodiments without departing from the underlying principles of the present technology. In some cases, well known structures and functions have not been shown or described in detail to avoid unnecessarily obscuring the description of the embodiments of the present technology. Although blocks of methods may be presented herein in a particular order, alternative embodiments may perform the blocks in a different order. Similarly, certain aspects of the present technology disclosed in the context of particular embodiments can be combined or eliminated in other embodiments. Furthermore, while advantages associated with certain embodiments of the present technology may have been disclosed in the context of those embodiments, other embodiments can also exhibit such advantages, and not all embodiments need necessarily exhibit such advantages or other advantages disclosed herein to fall within the scope of the technology. Accordingly, the disclosure and associated technology can encompass other embodiments not expressly shown or described herein, and the invention is not limited except as by the appended claims.

Throughout this disclosure, the singular terms “a,” “an,” and “the” include plural referents unless the context clearly indicates otherwise. Similarly, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising,” “including,” and “having” should be interpreted to mean including at least the recited feature(s) such that any greater number of the same feature and/or additional types of other features are not precluded.

Reference herein to “one embodiment,” “an embodiment,” “some embodiments” or similar formulations means that a particular feature, structure, operation, or characteristic described in connection with the embodiment can be included in at least one embodiment of the present technology. Thus, the appearances of such phrases or formulations herein are not necessarily all referring to the same embodiment. Furthermore, various particular features, structures, operations, or characteristics may be combined in any suitable manner in one or more embodiments.

Unless otherwise indicated, all numbers expressing concentrations, pressures, and other numerical values used in the specification and claims, are to be understood as being modified in all instances by the term “about.” As used herein, the use of relative terminology, such as “about”, “approximately”, “substantially” and the like refer to the stated value plus or minus ten percent. For example, the use of the term “about 100” refers to a range of from 90 to 110, inclusive. In instances in which the context requires otherwise and/or relative terminology is used in reference to something that does not include a numerical value, the terms are given their ordinary meaning to one skilled in the art. Accordingly, unless indicated to the contrary, the numerical parameters set forth in the following specification and attached claims are approximations that may vary depending upon the desired properties sought to be obtained by the present technology. At the very least, and not as an attempt to limit the application of the doctrine of equivalents to the scope of the claims, each numerical parameter should at least be construed in light of the number of reported significant digits and by applying ordinary rounding techniques. Additionally, all ranges disclosed herein are to be understood to encompass any and all subranges subsumed therein. For example, a range of “1 to 10” includes any and all subranges between (and including) the minimum value of 1 and the maximum value of 10, i.e., any and all subranges having a minimum value of equal to or greater than 1 and a maximum value of equal to or less than 10, e.g., 5.5 to 10.

The disclosure set forth above is not to be interpreted as reflecting an intention that any claim requires more features than those expressly recited in that claim. Rather, as the following claims reflect, inventive aspects lie in a combination of fewer than all features of any single foregoing disclosed embodiment. Thus, the claims following this Detailed Description are hereby expressly incorporated into this Detailed Description, with each claim standing on its own as a separate embodiment. This disclosure includes all permutations of the independent claims with their dependent claims.

I/We claim:

1. A method for optimizing a dosage rate of a diuretic provided to a patient during fluid therapy, the method comprising:
receiving data associated with a patient;
receiving an expected diuretic dosage rate for the patient from a model trained to compare the received data with historical data and/or training data associated with one or more other patients that have a generally similar treatment profile as the patient, wherein the model is configured to determine the expected diuretic dosage rate based on the received data and one or more historical diuretic dosage rates administered to the one or more other patients;
based on the expected diuretic dosage rate, causing a diuretic to be administered to the patient at a dosage rate; and
based, at least in part, on a response of the patient to the administration of the diuretic, adjusting the dosage rate of the diuretic.

2. The method of claim 1, wherein adjusting the dosage rate of the diuretic includes increasing or decreasing the dosage rate of the diuretic.

3. The method of claim 1, further comprising, prior to adjusting the dosage rate of the diuretic, providing a notification to a user including a suggested increase or decrease to the dosage rate of the diuretic.

4. The method of claim 1, wherein adjusting the diuretic dosage rate includes adjusting the diuretic dosage rate based, at least in part, on a difference between a measured urine output of the patient and a desired urine output of the patient.

5. The method of claim 1, wherein causing the diuretic to be administered includes causing an optimum diuretic to be administered, and wherein the method further comprises receiving the optimum diuretic from the model (i) trained to compare the received data with historical data and/or training data associated with one or more other patients that have an at least generally similar treatment profile as the patient and (ii) configured to determine the expected diuretic dosage rate based, at least in part, on the received data and one or more historical diuretics administered to the one or more other patients.

6. The method of claim 1, further comprising receiving, from the model a likelihood of success for one or more therapy escalation options including administration of an additional loop diuretic, temporary fluid matching, and/or thiazide administration, wherein the model is configured to compare the received data with historical data and/or training data associated with one or more other patients that have an at least generally similar treatment profile as the patient to determine the likelihood of success based, at least in part, on the received data and one or more historical instances and/or success rates for the one or more therapy escalation options administered to the one or more other patients.

7. A method associated with fluid levels of a fluid therapy system, the method comprising:
receiving data associated with a patient;
receiving an expected time until a quantity of a fluid in a container of the fluid therapy system meets a fluid quantity threshold from a model trained to compare the received data with historical data of one or more other patients that have a generally similar treatment profile as the patient, wherein the model is configured to determine the expected time based, at least in part, on the received data and one or more historical times for the one or more other patients; and
when the time is less than a threshold time, providing a notification to a user associated with the fluid and/or the container.

8. The method of claim 7, wherein the fluid includes a hydration fluid and the container includes a hydration fluid source.

9. The method of claim 7, wherein the fluid includes a diuretic and the container includes a diuretic source.

10. The method of claim 7, wherein the fluid includes urine and the container includes a collection container.

11. The method of claim 7, wherein the fluid quantity threshold is up to 25% of a volume of the container.

12. The method of claim 7, wherein providing the notification includes prompting the user to replace the container with another container.

13. The method of claim 7, wherein the container includes a hydration fluid source, and wherein providing the notification includes prompting the user to replace the hydration fluid source with a new hydration fluid source.

14. The method of claim 7, wherein the container includes a diuretic source, and wherein providing the notification includes prompting the user to replace the diuretic source.

15. The method of claim 7, wherein the container includes a urine collection container and wherein providing the notification includes prompting the user to empty the urine collection container.

16. A method for optimizing delivery of a hydration fluid to a patient during fluid therapy, the method comprising:
receiving data associated with a patient;
receiving a predicted hydration fluid infusion intolerance risk of the patient, wherein the predicted hydration fluid infusion intolerance risk is received from a model trained to compare the received data with historical data associated with one or more other patients that have a generally similar treatment profile as the patient, and wherein the model is configured to determine the predicted hydration fluid infusion intolerance risk based, at least in part, on the received data and one or more instances of hydration fluid infusion intolerance associated with the one or more other patients; and
based, at least in part, on the predicted hydration fluid infusion intolerance risk of the patient causing a hydration fluid to be administered to the patient at a hydration rate.

17. The method of claim 16, wherein the predicted hydration fluid infusion intolerance risk includes a likelihood that a hydration fluid infusion rate matching the patient's urine output increases a risk of one or more adverse events.

18. The method of claim 16, wherein the hydration rate is less than the patient's urine output rate.

19. The method of claim 16, wherein the hydration rate is greater than the patient's urine output rate.

20. The method of claim 16, wherein, if the predicted hydration fluid infusion intolerance risk is above a high risk threshold, no hydration fluid is administered or the hydration rate matches up to a first amount of the patient's urine output.

21. The method of claim 20, wherein the high risk threshold is at least 70%.

22. The method of claim 20, wherein if the predicted hydration fluid intolerance risk is at or below a low risk threshold, the hydration rate matches up to a second amount of the patient's urine output, greater than the first amount.

23. The method of claim 22, wherein the low risk threshold is up to 30%.

24. The method of claim 22, wherein, if the predicted hydration fluid infusion intolerance risk is between the low risk threshold and the high risk threshold, the hydration rate matches up to a third amount of the patient's urine output, between the first amount and the second amount.

25. The method of claim 22, wherein receiving data associated with the patient includes receiving urine profile data associated with a urine rate and/or a urine sodium content of the patient, wherein the model is trained to identify, within the historical data and/or training data associated with the one or more other patients, a subset of the one or more other patients that have historical urine profile data within a predetermined percentage of the received urine profile data.
